(19) United States
(12) Patent Application Publication
(10) Pub. No.: US 2024/0194335 A1
(43) Pub. Date: Jun. 13, 2024

(54) THERAPY SYSTEMS USING IMPLANT AND/OR BODY WORN MEDICAL DEVICES

(71) Applicants: COCHLEAR LIMITED, Macquarie University, NSW (AU); EPI-MINDER PTY LTD, East Melbourne, VIC (AU)

(72) Inventors: John Michael HEASMAN, Macquarie University, NSW (AU); Mark COOK, Melbourne, VIC (AU)

(21) Appl. No.: 18/285,305

(22) PCT Filed: Mar. 31, 2022
(86) PCT No.: PCT/IB2022/053033

§ 371 Date: Oct. 2, 2023

Related U.S. Application Data
(60) Provisional application No. 63/168,665, filed on Mar. 31, 2021.

(51) Int. Cl. G16H 40/67; A61B 5/00; A61M 5/142; A61M 5/172; A61N 1/05; G16H 20/30

(52) U.S. Cl. CPC G16H 40/67; A61B 5/4833; A61B 5/4839; A61M 5/14276; A61M 5/172; A61N 1/0541; G16H 20/30

(57) ABSTRACT

A system, including a body worn and/or implanted medical device, wherein the medical device is configured to receive and store data indicative of past treatment of a person who is the wearer and/or has been implanted with the medical device, and the medical device is configured to at least one of provide tissue stimulation to tissue of the person or sense phenomenon associated with the person.

### CROSS-REFERENCE TO RELATED APPLICATIONS

This application claims priority to U.S. Provisional Application No. 63/168,665, entitled THERAPY SYSTEMS USING IMPLANT AND/OR BODY WORN MEDICAL DEVICES, filed on Mar. 31, 2021, naming John Michael HEASMAN of Macquarie University, Australia as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

An exemplary embodiment includes a system, comprising a body worn and/or implanted medical device, wherein the medical device is configured to receive and store data indicative of past treatment of a person who is the wearer and/or has been implanted with the medical device, and the medical device is configured to at least one of provide tissue stimulation to tissue of the person or sense phenomenon associated with the person.

In an exemplary embodiment, there is a method, comprising receiving, indirectly or directly, from a body worn and/or implanted medical device, data indicative of at least one of a treatment history or a current medical condition of a person wearing and/or implanted with the medical device, evaluating at least the data and determining, based on the evaluation, a treatment regimen for the person, implementing the treatment regimen or providing the treatment regimen to the person.

In an exemplary embodiment, there is a closed-loop monitoring and therapeutic substance delivery system, wherein the closed-loop monitoring and therapeutic substance delivery system is configured to monitor one or more physiological parameters of a user of a medical device, and the closed-loop monitoring and therapeutic substance delivery system is further configured to use results from the monitored one or more physiological parameters to establish a dosage and/or schedule for future therapeutic substance(s) to be taken by and/or administered to the user.

In an exemplary embodiment, there is a method, comprising receiving an implanted hearing prosthesis recipient, obtaining data from a system of which the hearing prosthesis is apart while the recipient is received with the implant, the data relating prior treatment of the recipient and/or prior physiological data relating to the recipient, obtaining a dosage and/or schedule for a therapeutic substance to be used by the recipient, wherein the obtained dosage and/or schedule is based at least in part on the obtained data, and providing the dosage and/or schedule to the recipient.

In an exemplary embodiment, there is a system, comprising a hearing prosthesis, including one of an electrode based stimulation system or a mechanical actuator based stimulation system and an RF receiver configured to receive data indicative of past treatment of a person who is the recipient of the hearing prosthesis and including random access memory in which the data indicative of past treatment of the person is stored.

In an exemplary embodiment, there is a system, comprising a blood chemistry monitoring prosthesis, including a needle configured to extend into skin of a human to access blood of the human an RF receiver configured to receive data indicative of past treatment of a person who is the recipient of the hearing prosthesis and including random access memory in which the data indicative of past treatment of the person is stored.

### BRIEF DESCRIPTION OF THE DRAWINGS

Embodiments are described below with reference to the attached drawings, in which:

FIG. 1 is a perspective view of an exemplary hearing prosthesis in which at least some of the teachings detailed herein are applicable;

FIG. 2 presents a functional block diagram of an example cochlear implant;

FIG. 3A and FIG. 3B present exemplary systems according to some embodiments;

FIG. 4 presents an exemplary external component;

FIGS. 5 and 6 and 7 present schematics of some exemplary body monitoring systems;

FIG. 8 presents an exemplary sensory prosthesis;

FIG. 9 present at least an exemplary monitoring device;

FIGS. 10 to 14 present exemplary monitoring devices and exemplary therapeutic substance delivery devices;

FIG. 15 presents an exemplary flowchart for an exemplary algorithm;

FIGS. 16 and 17 present exemplary systems;

FIGS. 18-20 present exemplary monitoring devices;

FIG. 21 presents an exemplary flowchart for an exemplary algorithm; and

FIGS. 22 and 23 present communication and computing examples with which embodiments can be used.

### DETAILED DESCRIPTION

Merely for ease of description, the techniques presented herein are primarily described herein with reference to an illustrative medical device, namely a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used with other hearing prostheses, including acoustic hearing aids, bone conduction devices, middle ear auditory prostheses, direct acoustic stimulators, other electrically simulating auditory prostheses (e.g., auditory brain stimulators), etc. The techniques presented herein may also be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc. In further embodiments, the techniques presented herein may be used with air purifiers or air sensors (e.g., automatically adjust depending on environment), hospital beds, identification (ID) badges/bands, or other hospital equipment or instruments.

The teachings detailed herein are implemented in sensory prostheses, such as hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. Moreover, with respect to any teachings herein, such corresponds to a disclosure of utilizing those teachings with all of or parts of a cochlear implant, a bone conduction device (active and passive transcutaneous bone conduction devices, and percutaneous bone conduction devices) and a middle ear implant, providing that the art enables such, unless otherwise noted. To be clear, any teaching herein with respect to a specific sensory prosthesis corresponds to a disclosure of utilizing those teachings in/with any of the aforementioned hearing prostheses, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

Thus, merely for ease of description, the first illustrative medical device is a hearing prosthesis. Any techniques presented herein described for one type of hearing prosthesis or any other device disclosed herein corresponds to a disclosure of another embodiment of using such teaching with another device (and/or another type of hearing device including other types of bone conduction devices (active transcutaneous and/or passive transcutaneous), middle ear auditory prostheses (particularly, the EM vibrator/actuator thereof), direct acoustic stimulators), etc. The techniques presented herein can be used with implantable/implanted microphones (where such is a transducer that receives vibrations and outputs an electrical signal (effectively, the reverse of an EM actuator), whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), and thus any disclosure herein is a disclosure of utilizing such devices with the teachings herein (and vice versa), providing that the art enables such. The teachings herein can also be used with conventional hearing devices, such as telephones and ear bud devices connected MP3 players or smart phones or other types of devices that can provide audio signal output, that use an EM transducer. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

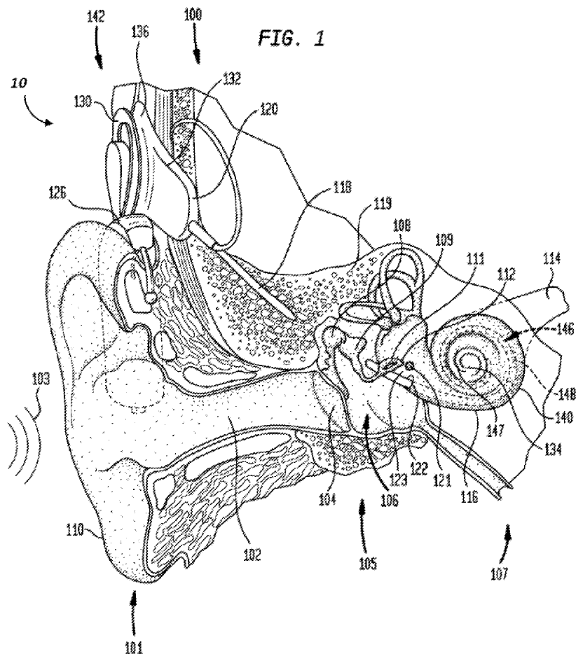

FIG. 1 is a perspective view of a cochlear implant, referred to as cochlear implant 100, implanted in a recipient, to which some embodiments detailed herein and/or variations thereof are applicable. The cochlear implant 100 is part of a system 10 that can include external components in some embodiments, as will be detailed below. Additionally, it is noted that the teachings detailed herein are also applicable to other types of hearing prostheses, such as, by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing.

In view of the above, it is to be understood that at least some embodiments detailed herein and/or variations thereof are directed towards a body-worn sensory supplement medical device (e.g., the hearing prosthesis of FIG. 1, which supplements the hearing sense, even in instances when there are no natural hearing capabilities, for example, due to degeneration of previous natural hearing capability or to the lack of any natural hearing capability, for example, from birth). It is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities and to recipients having no natural vision capabilities). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

Thus, as seen above, one variety of implanted devices depends on an external component to provide certain functionality and/or power. For example, the recipient of the implanted device can wear an external component that provides power and/or data (e.g., a signal representative of sound) to the implanted portion that allow the implanted device to function. In particular, the implanted device can lack a battery and can instead be totally dependent on an external power source providing continuous power for the implanted device to function. Although the external power source can continuously provide power, characteristics of the provided power need not be constant and may fluctuate. Additionally, where the implanted device is an auditory prosthesis such as a cochlear implant, the implanted device can lack its own sound input device (e.g., a microphone). It is sometimes utilitarian to remove the external component. For example, it is common for a recipient of an auditory prosthesis to remove an external portion of the prosthesis while sleeping. Doing so can result in loss of function of the implanted portion of the prosthesis, which can make it impossible for recipient to hear ambient sound. This can be less than utilitarian and can result in the recipient being unable to hear while sleeping. Loss of function would also prevent the implanted portion from responding to signals representative of streamed content (e.g., music streamed from a phone) or providing other functionality, such as providing tinnitus suppression noise.

The external component that provides power and/or data can be worn by the recipient, as detailed above. While a wearable external device is worn by a recipient, the external device is typically in very close proximity and tightly aligned with an implanted component. The wearable external device can be configured to operate in these conditions. Conversely, in some instances, an unworn device can generally be further away and less tightly aligned with the implanted component. This can create difficulties where the implanted device depends on an external device for power and data (e.g., where the implanted device lacks its own battery and microphone), and the external device can need to continuously and consistently provide power and data in order to allow for continuous and consistent functionality of the implanted device.

Technologies disclosed herein can be used to provide power to and/or data to and/or retrieve data from an implantable device in situations where a recipient is not wearing an external device. The technologies can overcome one or more challenges associated therewith. In an example, disclosed technologies can provide a source of power and/or data for an implanted medical device via a system that includes a pillow or other headrest or other bodyrest component (mattress, blanket, etc.). Disclosed technologies can be configured to continuously and/or intermittently provide power and data to an implantable medical device over a period of time (e.g., substantially the entire period of time where the recipient is resting their head on the pillow). Characteristics of the continuously provided power need not be constant. For example, the power may fluctuate because the efficiency of the link between the implant and the pillow may vary as the recipient's head moves, causing the proximity of the coils to vary. The power to the implanted electronics can be smoothed for example using tank capacitors. It is common for recipients of an implanted medical device to remove their external devices while sleeping and during that time pillows are often placed in close proximity to the implanted prosthesis. In particular, auditory implants are typically disposed in close proximity to a recipient's ears and people typically place their head on a pillow such that one or both ears are close to the pillow. Thus, it can be utilitarian to incorporate a pillow into a system for providing functionality of a worn external device while a recipient of an implantable device is sleeping. For a recipient of bilateral auditory implants, it may be sufficient for nighttime use for only one of the two devices to function. For instance, a first device being closest to the pillow may receive sufficient power and/or data to function while a second device that is further away from the pillow may receive insufficient power and/or data to function.

Reference may be made herein to pillows or other headrests for concision, but disclosed technologies can be used in conjunction with a variety of articles. Headrests can include, for example, pillows, cushions, pads, head supports, and mattresses, among others. Such articles may be covered (e.g., with a pillowcase) or uncovered. Additionally, the disclosed external system components can be used with any of a variety of systems in accordance with embodiments of the technology. For example, in many embodiments, the technology is used in conjunction with a conventional cochlear implant system. FIG. 1 depicts an exemplary cochlear implant system that can benefit from use with technology disclosed herein.

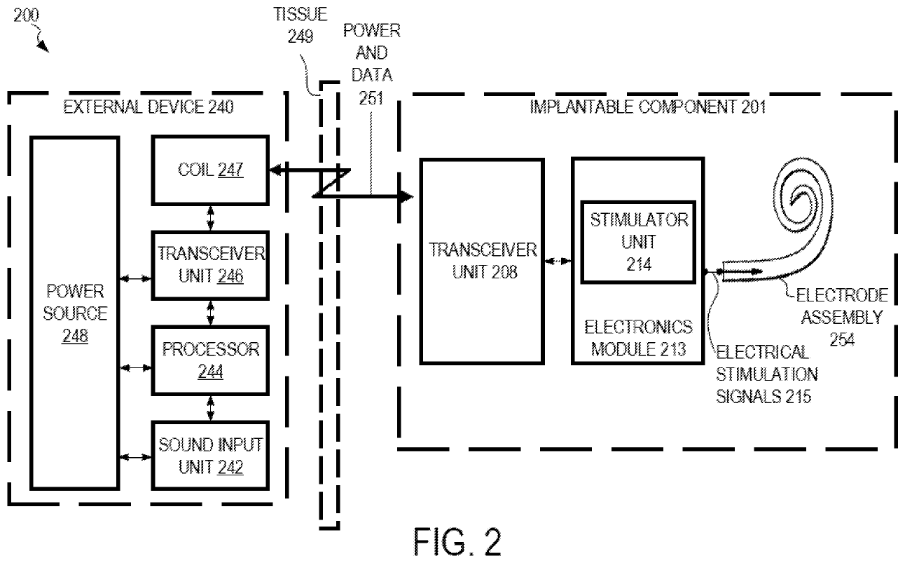

FIG. 2 is a functional block diagram of a cochlear implant system 200 that can benefit from the use of a pillow system in accordance with certain examples of the technology described herein. The cochlear implant system 200 includes an implantable component 201 (e.g., implantable component 100 of FIG. 1) configured to be implanted beneath a recipient's skin or other tissue 249, and an external device 240 (e.g., the external device 142 of FIG. 1).

The external device 240 can be configured as a wearable external device, such that the external device 240 is worn by a recipient in close proximity to the implantable component, which can enable the implantable component 201 to receive power and stimulation data from the external device 240. As described in FIG. 1, magnets can be used to facilitate an operational alignment of the external device 240 with the implantable component 201. With the external device 240 and implantable component 201 in close proximity, the transfer of power and data can be accomplished through the use of near-field electromagnetic radiation, and the components of the external device 240 can be configured for use with near-field electromagnetic radiation.

Implantable component 201 can include a transceiver unit 208, electronics module 213, which module can be a stimulator assembly of a cochlear implant, and an electrode assembly 254 (which can include an array of electrode contacts disposed on lead 118 of FIG. 1). The transceiver unit 208 is configured to transcutaneously receive power and/or data from external device 240. As used herein, transceiver unit 208 refers to any collection of one or more components which form part of a transcutaneous energy transfer system. Further, transceiver unit 208 can include or be coupled to one or more components that receive and/or transmit data or power. For example, the example includes a coil for a magnetic inductive arrangement coupled to the transceiver unit 208. Other arrangements are also possible, including an antenna for an alternative RF system, capacitive plates, or any other utilitarian arrangement. In an example, the data modulates the RF carrier or signal containing power. The transcutaneous communication link established by the transceiver unit 208 can use time interleaving of power and data on a single RF channel or band to transmit the power and data to the implantable component 201. In some examples, the processor 244 is configured to cause the transceiver unit 246 to interleave power and data signals, such as is described in U.S. Patent Publication Number 2009/0216296 to Meskens. In this manner, the data signal is modulated with the power signal, and a single coil can be used to transmit power and data to the implanted component 201. Various types of energy transfer, such as infrared (IR), electromagnetic, capacitive and inductive transfer, can be used to transfer the power and/or data from the external device 240 to the implantable component 201.

Aspects of the implantable component 201 can require a source of power to provide functionality, such as receive signals, process data, or deliver electrical stimulation. The source of power that directly powers the operation of the aspects of the implantable component 201 can be described as operational power. There are two exemplary ways that the implantable component 201 can receive operational power: a power source internal to the implantable component 201 (e.g., a battery) or a power source external to the implantable component. However, other approaches or combinations of approaches are possible. For example, the implantable component may have a battery but nonetheless receive operational power from the external component (e.g., to preserve internal battery life when the battery is sufficiently charged).

The internal power source can be a power storage element (not pictured). The power storage element can be configured for the long-term storage of power, and can include, for example, one or more rechargeable batteries. Power can be received from an external source, such as the external device 240, and stored in the power storage element for long-term use (e.g., charge a battery of the power storage element). The power storage element can then provide power to the other components of the implantable component 201 over time as needed for operation without needing an external power source. In this manner, the power from the external source may be considered charging power rather than operational power because the power from the external power source is for charging the battery (which in turn provides operational power) rather than for directly powering aspects of the implantable component 201 that require power to operate. The power storage element can be a long-term power storage element configured to be a primary power source for the implantable component 201.

In some embodiments, the implantable component 201 receives operational power from the external device 240 and the implantable component 201 does not include an internal power source (e.g., a battery)/internal power storage device. In other words, the implantable component 201 is powered solely by the external device 240 or another external device, which provides enough power to the implantable component 201 to allow the implantable component to operate (e.g., receive data signals and take an action in response). The operational power can directly power functionality of the device rather than charging a power storage element of the external device implantable component 201. In these examples, the implantable component 201 can include incidental components that can store a charge (e.g., capacitors) or small amounts of power, such as a small battery for keeping volatile memory powered or powering a clock (e.g., motherboard CMOS batteries). But such incidental components would not have enough power on their own to allow the implantable component to provide primary functionality of the implantable component 201 (e.g., receiving data signals and taking an action in response thereto, such as providing stimulation) and therefore cannot be said to provide operational power even if they are integral to the operation of the implantable component 201.

As shown, electronics module 213 includes a stimulator unit 214 (e.g., which can correspond to the stimulator of FIG. 1). Electronics module 213 can also include one or more other components used to generate or control delivery of electrical stimulation signals 215 to the recipient. As described above with respect to FIG. 1, a lead (e.g., elongate lead 118 of FIG. 1) can be inserted into the recipient's cochlea. The lead can include an electrode assembly 254 configured to deliver electrical stimulation signals 215 generated by the stimulator unit 214 to the cochlea.

In the example system 200 depicted in FIG. 2, the external device 240 includes a sound input unit 242, a sound processor 244, a transceiver unit 246, a coil 247, and a power source 248. The sound input unit 242 is a unit configured to receive sound input. The sound input unit 242 can be configured as a microphone (e.g., arranged to output audio data that is representative of a surrounding sound environment), an electrical input (e.g., a receiver for a frequency modulation (FM) hearing system), and/or another component for receiving sound input. The sound input unit 242 can be or include a mixer for mixing multiple sound inputs together.

The processor 244 is a processor configured to control one or more aspects of the system 200, including converting sound signals received from sound input unit 242 into data signals and causing the transceiver unit 246 to transmit power and/or data signals. The transceiver unit 246 can be configured to send or receive power and/or data 251. For example, the transceiver unit 246 can include circuit components that send power and data (e.g., inductively) via the coil 247. The data signals from the sound processor 244 can be transmitted, using the transceiver unit 246, to the implantable component 201 for use in providing stimulation or other medical functionality.

The transceiver unit 246 can include one or more antennas or coils for transmitting the power or data signal, such as coil 247. The coil 247 can be a wire antenna coil having of multiple turns of electrically insulated single-strand or multi-strand wire. The electrical insulation of the coil 247 can be provided by a flexible silicone molding. Various types of energy transfer, such as infrared (IR), radiofrequency (RF), electromagnetic, capacitive and inductive transfer, can be used to transfer the power and/or data from external device 240 to implantable component 201.

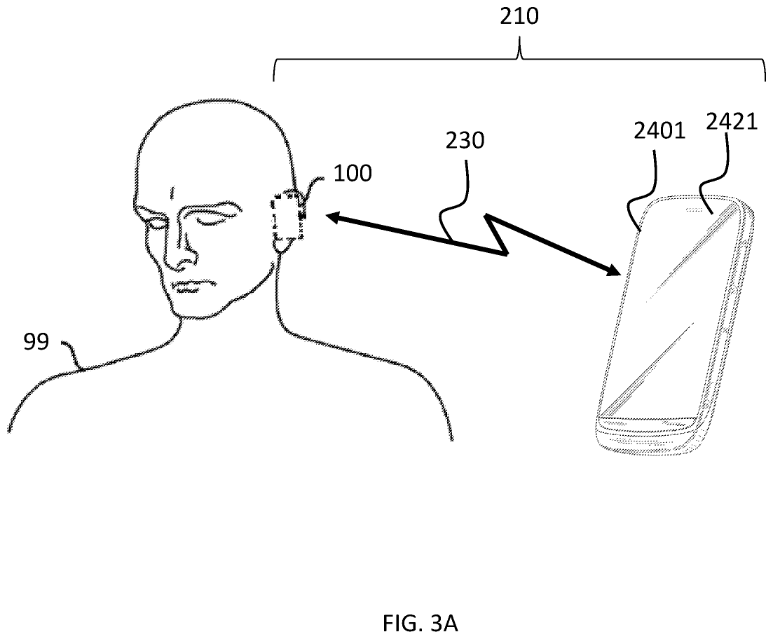

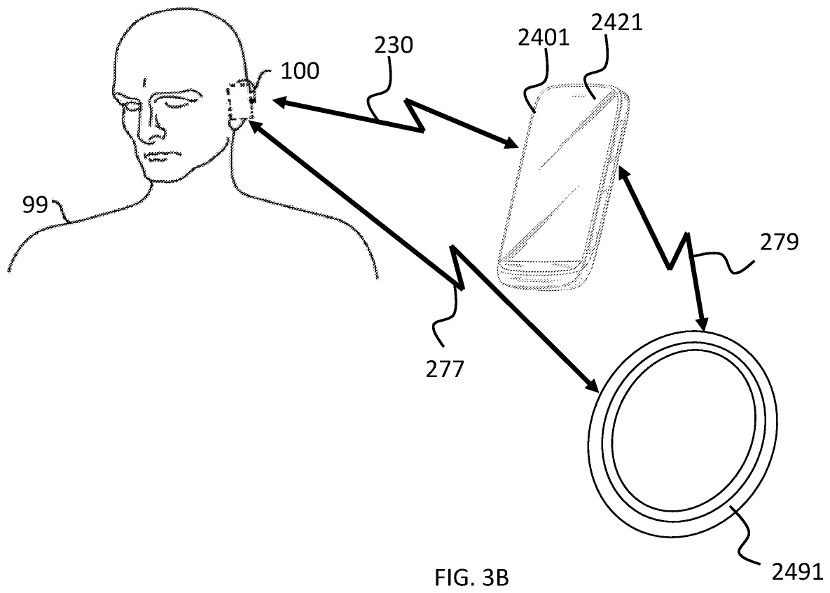

FIG. 3A depicts an exemplary system 210 according to an exemplary embodiment, including hearing prosthesis 100, which, in an exemplary embodiment, corresponds to cochlear implant 100 detailed above, and a portable body carried device (e.g. a portable handheld device as seen in FIG. 2A, a watch, a pocket device, etc.) 2401 in the form of a mobile computer having a display 2421. The system includes a wireless link 230 between the portable handheld device 2401 and the hearing prosthesis 100. In an embodiment, the prosthesis 100 is an implant implanted in recipient 99 (represented functionally by the dashed lines of box 100 in FIG. 3A).

In an exemplary embodiment, the system 210 is configured such that the hearing prosthesis 100 and the portable handheld device 2401 have a symbiotic relationship. In an exemplary embodiment, the symbiotic relationship is the ability to display data relating to, and, in at least some instances, the ability to control, one or more functionalities of the hearing prosthesis 100. In an exemplary embodiment, this can be achieved via the ability of the handheld device 2401 to receive data from the hearing prosthesis 100 via the wireless link 230 (although in other exemplary embodiments, other types of links, such as by way of example, a wired link, can be utilized). As will also be detailed below, this can be achieved via communication with a geographically remote device in communication with the hearing prosthesis 100 and/or the portable handheld device 2401 via link, such as by way of example only and not by way of limitation, an Internet connection or a cell phone connection. In some such exemplary embodiments, the system 210 can further include the geographically remote apparatus as well. Again, additional examples of this will be described in greater detail below.

As noted above, in an exemplary embodiment, the portable handheld device 2401 comprises a mobile computer and a display 2421. In an exemplary embodiment, the display 2421 is a touchscreen display. In an exemplary embodiment, the portable handheld device 2401 also has the functionality of a portable cellular telephone. In this regard, device 2401 can be, by way of example only and not by way of limitation, a smart phone, as that phrase is utilized generically. That is, in an exemplary embodiment, portable handheld device 2401 comprises a smart phone, again as that term is utilized generically.

It is noted that in some other embodiments, the device 2401 need not be a computer device, etc. It can be a lower tech recorder, or any device that can enable the teachings herein.

The phrase “mobile computer” entails a device configured to enable human-computer interaction, where the computer is expected to be transported away from a stationary location during normal use. Again, in an exemplary embodiment, the portable handheld device 2401 is a smart phone as that term is generically utilized. However, in other embodiments, less sophisticated (or more sophisticated) mobile computing devices can be utilized to implement the teachings detailed herein and/or variations thereof. Any device, system, and/or method that can enable the teachings detailed herein and/or variations thereof to be practiced can be utilized in at least some embodiments. (As will be detailed below, in some instances, device 2401 is not a mobile computer, but instead a remote device (remote from the hearing prosthesis 100. Some of these embodiments will be described below).)

In an exemplary embodiment, the portable handheld device 2401 is configured to receive data from a hearing prosthesis and present an interface display on the display from among a plurality of different interface displays based on the received data. Exemplary embodiments will sometimes be described in terms of data received from the hearing prosthesis 100. However, it is noted that any disclosure that is also applicable to data sent to the hearing prosthesis from the handheld device 2401 is also encompassed by such disclosure, unless otherwise specified or otherwise incompatible with the pertinent technology (and vice versa).

It is noted that in some embodiments, the system 210 is configured such that cochlear implant 100 and the portable device 2401 have a relationship. By way of example only and not by way of limitation, in an exemplary embodiment, the relationship is the ability of the device 2401 to serve as a remote microphone for the prosthesis 100 via the wireless link 230. Thus, device 2401 can be a remote mic. That said, in an alternate embodiment, the device 2401 is a stand-alone recording/sound capture device.

It is noted that in at least some exemplary embodiments, the device 2401 corresponds to an Apple Watch™ Series 1 or Series 2, as is available in the United States of America for commercial purchase as of Jan. 10, 2021. In an exemplary embodiment, the device 2401 corresponds to a Samsung Galaxy Gear™ Gear 2, as is available in the United States of America for commercial purchase as of Jan. 10, 2021. The device is programmed and configured to communicate with the prosthesis and/or to function to enable the teachings detailed herein.

In an exemplary embodiment, a telecommunication infrastructure can be in communication with the hearing prosthesis 100 and/or the device 2401. By way of example only and not by way of limitation, a telecoil 2491 or some other communication system (Bluetooth, etc.) is used to communicate with the prosthesis and/or the remote device. FIG. 2B depicts an exemplary quasi-functional schematic depicting communication between an external communication system 2491 (e.g., a telecoil), and the hearing prosthesis 100 and/or the handheld device 2401 by way of links 277 and 279, respectively (note that FIG. 3B depicts two-way communication between the hearing prosthesis 100 and the external audio source 2491, and between the handheld device and the external audio source 2491—in alternate embodiments, the communication is only one way (e.g., from the external audio source 2491 to the respective device)). It is noted that unless otherwise noted, the embodiment of FIG. 3B is applicable to any body worn medical device/implanted device disclosed herein in some embodiments.

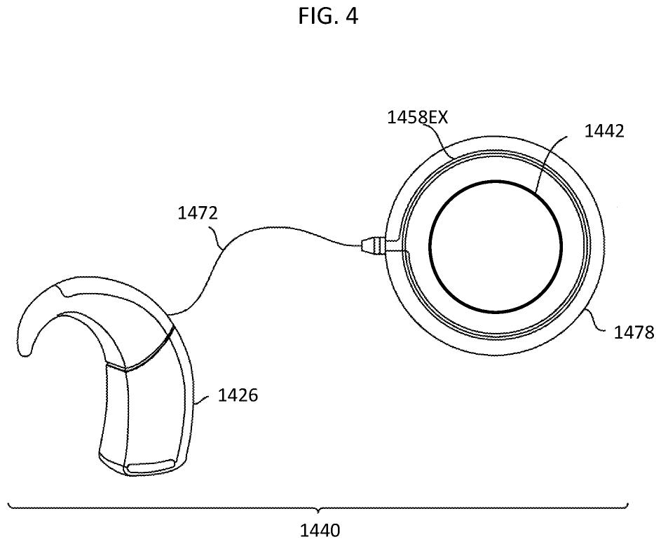

It is noted that while some embodiments detailed herein are described in terms of utilizing an external device that is fixed or otherwise relatively immobile (e.g., a device integrated into a bed, for example) or a device that can be in a relatively easily movable object (a pillow, a shirt, etc.), to communicate and/or power the implanted component, it is to be understood that these devices can also be powered by their traditional external components and/or communicated therewith via their traditional external components. In this regard, FIG. 4 depicts an exemplary external component 1440. External component 1440 can correspond to external component 142 of the system 10 (it can also represent other body worn devices herein/devices that are used with implanted portions). As can be seen, external component 1440 includes a behind-the-ear (BTE) device 1426 which is connected via cable 1472 to an exemplary headpiece 1478 including an external inductance coil 1458EX, corresponding to the external coil of FIG. 1. As illustrated, the external component 1440 comprises the headpiece 1478 that includes the coil 1458EX and a magnet 1442. This magnet 1442 interacts with the implanted magnet (or implanted magnetic material) of the implantable component to hold the headpiece 1478 against the skin of the recipient. In an exemplary embodiment, the external component 1440 is configured to transmit and/or receive magnetic data and/or transmit power transcutaneously via coil 1458EX to the implantable component, which includes an inductance coil. The coil 1458X is electrically coupled to BTE device 1426 via cable 1472. BTE device 1426 may include, for example, at least some of the components of the external devices/components described herein.

Accordingly, in an exemplary embodiment, external component 1440 can be utilized with the implantable component that is an implantable hearing prosthesis and/or an implantable retinal implant and/or an implantable sense prosthesis as detailed herein where the implanted coil is implanted near or in the head.

In some embodiments, with respect to any of the devices detailed herein and/or variations thereof, there can be utilitarian value with respect to measuring/detecting a physiological feature of the user. In the case of cochlear implants, in an exemplary embodiment, the electrically evoked compound action potential in response to stimulating the cochlea can be measured. In another example, the EEG of the patient/recipient is measured. Many physiological and environmental factors can influence recordings. There is benefit in understanding the factors when measuring a physiological feature of a user/recipient.

In an exemplary embodiment, there can be sensors, such as implanted or internal sensors, that can be utilitarian in at least partially aiding in a process that includes determining a temporal period when it might be utilitarian to take a measurement of something associated with a person. By way of example only and not by way of limitation, in an embodiment where there is the execution of acoustic probing (e.g., via the utilization of any of the hearing prostheses disclosed herein that can enable such, including a conventional hearing aid, or the utilization of a non-prosthetic device, such as the speaker of a smart phone or smart device, etc.) to obtain data related to a recipient based on a reaction or a response to a probe or any other utilitarian phenomenon that can be detected, for example, a microphone could be used to obtain data that can be used to ascertain, directly and/or through latent variables, that there exists an environment where external noise is at a level and/or below a level such that the use of the acoustic probe and/or the data resulting from such utilization can be used with a minimum of efficacy (e.g., that the person can hear the sound generated by the acoustic probe vs. the ambient noise). In an exemplary embodiment, acoustic probing is executed using an acoustic-type signal and/or an acoustic simulating signal, while in other embodiments, the acoustic signal is a purely acoustic signal, while in other embodiments, the acoustic-type signal excludes a purely acoustic signal. In some embodiments, the probing is probing using electrical stimulation of tissue, etc.

In view of the above, there is an apparatus, comprising a medical device, such as any of the medical devices disclosed herein, such as, for example, the cochlear implant above and/or a conventional hearing aid, or a retinal implant, etc. In this exemplary embodiment, the medical device is configured to determine whether or not a data collection activity should be commenced, wherein the data is physiological data associated with a recipient of the medical device. In an exemplary embodiment where the medical device is a hearing prosthesis, the hearing prosthesis is configured to evaluate a sound environment of the hearing prosthesis to determine whether or not the data collection activity should be commenced. By way of example only and not by way of limitation, in an exemplary embodiment, there is the utilization of a hearing prosthesis in accordance with at least some of the teachings detailed herein, in conjunction with data collection associated with recording electroencephalogram (EEG) data. Indeed, in an exemplary embodiment, an acoustic transducer, or any other sound creating or a hearing percept evoking device that can be utilized, is utilized to evoke a hearing percept. By way of example only and not by way of limitation, this can be done utilizing an implanted actuator, such as a bone conduction device, or a middle ear implant. Still further, this can be done utilizing a cochlear implant, an auditory brainstem implant, or an auditory midbrain implant, etc. In an alternate embodiment, a conventional hearing aid can be utilized to evoke the hearing percept. In some embodiments, a combination of two or more of the aforementioned devices can be utilized to evoke the hearing percept. Other devices can be utilized as well. In an exemplary embodiment, the above-noted portable handheld device 2401 can be utilized, or a noise or sound producing device specifically designed and fabricated for medical procedures. In an exemplary embodiment, the determination is made based on whether the recipient is moving or not, while in other embodiments, the movement is determined based on non-movement data/data that is not related to movement data (e.g., data that is not based on the output of an accelerometer and/or a device that determines that the recipient is moving, as different from a device that can determine that the recipient has moved/is in a new location than that which was previously the case). To be clear, “non-movement data” as used herein means that the data is unrelated to movement, not that the recipient is not moving.

In many instances, the teachings detailed herein will be directed towards implantable components and the like and/or prostheses. It is noted that any disclosure herein of an implantable component corresponds to an alternative disclosure of an apparatus or a component that is not implanted that has the functionality that is the same as or sufficiently efficaciously similar to the implanted component. Further, any disclosure herein of a prosthesis corresponds to an alternative disclosure of an apparatus or a component that is not a prostatic component. Any disclosure herein of a prosthesis corresponds to an alternative disclosure of a body worn or body carried device. Note further, any disclosure of a body worn or body carried device and/or a prosthesis and/or an implanted component corresponds to a disclosure of a device that is stationary or semi-stationary that has that functionality. All of this is contingent upon the art enabling such, as well as any explicit proviso detailed herein stating that such is not the case.

In many instances, implanted devices, such as implanted electrodes, are disclosed herein for utilization with respect to monitoring physiological characteristics. Consistent with the aforementioned statements in the above paragraph, any disclosure herein of an implanted component that is utilized for measurements or sensation purposes also corresponds to an alternate disclosure of a device and/or an apparatus for component that is not implanted but has that functionality or otherwise enable such functionality, again subject to the aforementioned provisos.

In this exemplary embodiment under discussion, the sound that is produced or otherwise the stimulus that is utilized to evoke a hearing percept are utilized to elicit a time specific response in the brain, such as an EEG response. In this regard, sound, or the perceptive sound, can cause the brain to be stimulated and thus produce brain waves which can be detected/recorded and the data associated therewith can be analyzed, sometimes in real time, to evaluate the state of a person's brain.

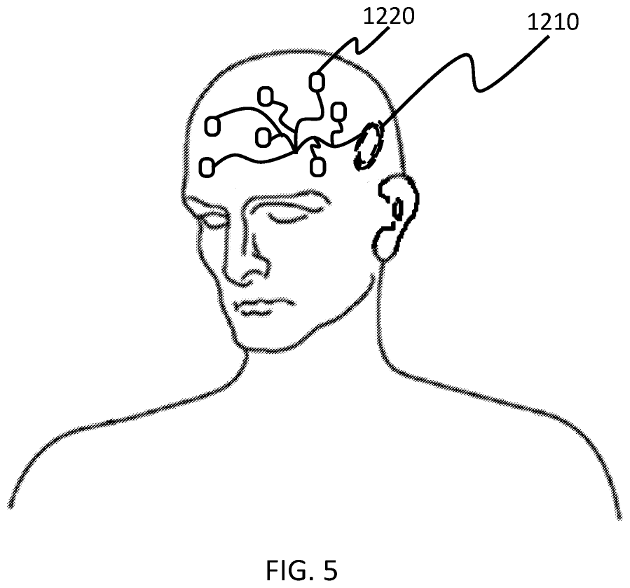

FIG. 5 provides an exemplary embodiment of an EEG system that is implanted in the recipient, where read/sense electrodes 1220 are arrayed inside a recipient's head and in signal communication with a coil 1210 via electrical leads. In this embodiment, the implanted device has no recording/storage capabilities, and requires an external device to receive a signal from the implanted inductance coil 1210 so as to retrieve in real time the signal therefrom. Not shown is an implantable component that converts the electricity sensed by the sensor/read electrodes into a signal that is transmitted by the inductance coil 1210. In an exemplary embodiment, the sensor arrangement seen in FIG. 5 is an implanted EEG sensor arrangement.

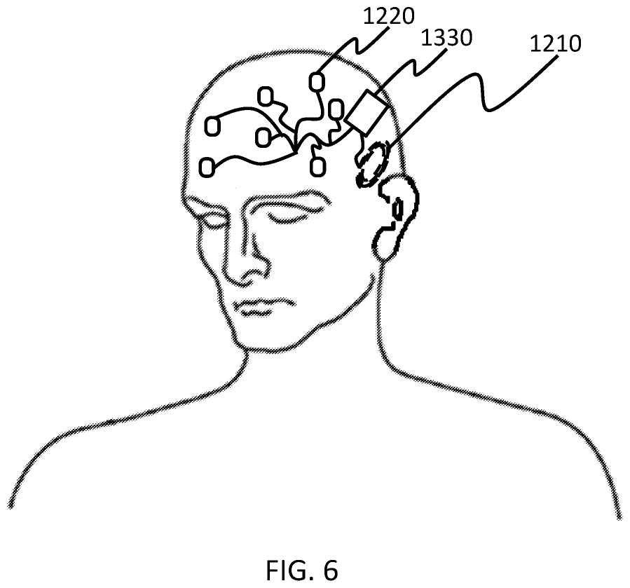

FIG. 6 depicts another arrangement of an implantable sensor arrangement that again includes the sensor/read electrodes 1220 and the leads. Here, in this embodiment, there is a housing 1330 which includes circuitry that is configured to receive the signals from the leads from the electrodes 1220 and record the data therefrom or otherwise store the data, and permits the data to be periodically read from an external device when the external device comes into signal communication with the implanted inductance coil 1210. Alternatively, and/or in addition to this, the circuitry is configured to periodically energize the inductance coil 1210 so as to provide the data to the coil 1210 so that it creates an inductance signal which in turn communicates with an external component that reads the signal and thus reads the data associated with the electrodes. Thus, in at least some exemplary embodiments, the implantable apparatus is configured to stream the data. Still further, in some embodiments, the data is not streamed, but instead provided in bursts.

Any arrangement that can enable the data associated with the read electrodes to be provided from inside the recipient to outside the recipient can be utilized in at least some exemplary embodiments. In this regard, traditional implanted EEG sensor arrangements can be obtained and modified so as to implement the teachings detailed herein and/or variations thereof.

It is noted that some embodiments of the sensor arrangement of FIG. 5 or 6 include an implanted battery or otherwise implanted power storage arrangement, while in other embodiments the arrangement specifically does not.

In view of the above, it is to be understood that in at least some exemplary embodiments, there are traditional implanted EEG and EKG sensor systems that are configured to communicate with the external devices detailed herein (e.g., the device of FIG. 4, or the aforementioned “pillow chargers” or “bed chargers,” etc.). In an exemplary embodiment, the structure implanted in the recipient is the exact same thing as these traditional sensor systems, with the exception that they have been modified to operate in the various modes detailed herein, such as by way of programming or by structural modification or by the inclusion of logic circuitry, etc. That is, in an exemplary embodiment, the sensory systems of FIGS. 5 and 6 are used in combination with the pillow charger detailed above for communication and/or powering and/or charging. Any disclosure herein of the use of the pillow charger associated with the hearing prosthesis detailed above also corresponds to the use of the pillow charger for data transfer and/or for powering and/or charging the sensor systems of FIGS. 5 and 6 or any other sensor systems detailed herein, just as any disclosure associated with the pillow charger vis-à-vis the cochlear implant also corresponds to a disclosure of such with respect to an implanted middle ear prosthesis, a DACI and an active transcutaneous bone conduction device.

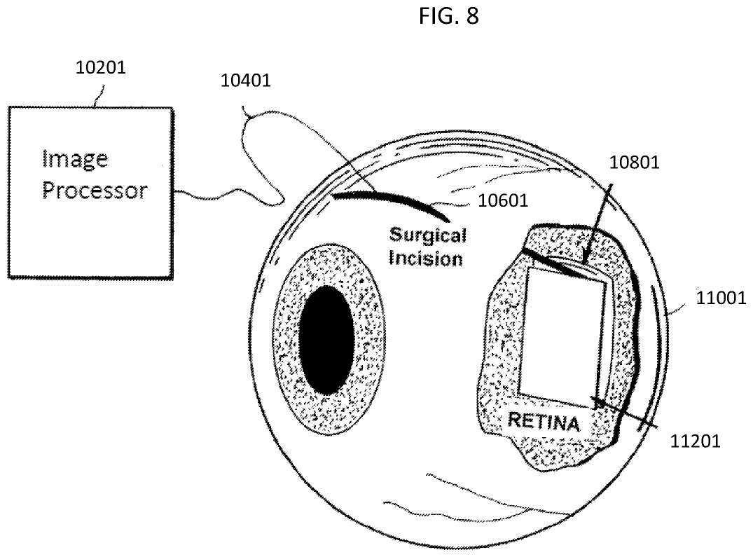

FIG. 8 presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

In an exemplary embodiment, there is a system, comprising, a body worn and/or implanted medical device, such as for example the implants described above, or other types of medical devices, such as, for example a blood glucose monitoring device, additional details of which will be described below. In this exemplary embodiment, the medical device is configured to receive and store data indicative of past treatment of a person who is the wearer and/or implanted with the medical device. Further, the medical device is configured to at least one of provide tissue stimulation to tissue of the person or sense phenomena associated with the person, which includes devices that do both. For example, the cochlear implant of FIG. 1 can include any one or more of the features detailed in U.S. Patent Application Publication No. 2012/0316454 to inventor Paul Carter. For example, that patent application publication details the utilization of electrode impedance spectroscopy to evaluate various physiological features associated with the cochlea, where the electrodes are the electrodes that are also utilized to provide stimulation to evoke a hearing percept in other uses of that implant. Further by example, PCT Application Publication No. WO 2019/186373, to inventor John Michael Heasman, details the utilization of electrical techniques for biomarker detection in a cochlea, which could be implemented in the medical device at issue in some embodiments. Still further, the medical device could be a device different than a cochlear implant, such as a middle ear implant, which implant could utilize some or all of the techniques detailed in U.S. Patent Application Publication No. 2017/0347209, to Inventor John Michael Heasman. And while the aforementioned exemplary patent application publications combine a cochlear implant or other hearing prostheses with sensing capabilities, in other embodiments, the body worn and/or implanted medical device can simply be a device that senses, as opposed to provides, stimulation, but of course, consistent with the aforementioned publications, the medical device can do both in some embodiments. In further embodiment, this does not preclude a scenario where a person has a stimulation device and a separate sensing device. Indeed, many embodiments disclosed herein specifically and purposely physically bifurcate and/or trifurcate, etc., the various components.

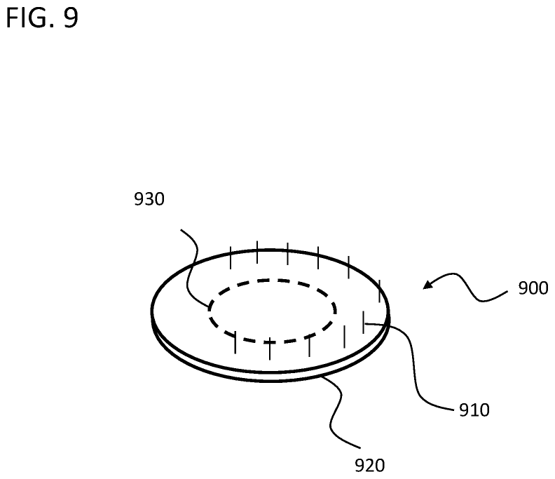

The medical device could be a retinal implant, which implant is configured to evaluate the level of stimulation that is experienced by the recipient owing to the device (which could be utilitarian in predicting a potential seizure, for example, owing to the stimulation). The medical device could also be a transdermal patch of FIG. 9 that is configured to monitor blood sugar levels, or other phenomena. The medical device could also be the EEG system of FIG. 5 or 6, etc. Note also that in some embodiments, that EEG system could also provide stimulation, and in alternative embodiments, the system could instead be a system that provides stimulation without a sensor. And while the arrangements of FIG. 5, etc., are presented in terms of implantable systems, in an alternate embodiment, the electrodes and the entire system could be instead an external/body worn medical device. The electrodes can be located on the surface of the skin.

Moreover, returning back to the cochlear implant and/or the implanted hearing prosthesis systems, it is noted that embodiments can also include separate systems associated with those implants that utilize different principles of operation from that associated with how those devices provide stimulation to sense phenomenon associated with the person. In an exemplary embodiment, the cochlear implant could also have a component that senses a physiological phenomenon associated with the perilymph utilizing a sensor completely separate and unrelated to the electrodes that are utilized for stimulating the cochlea.

Returning back to the original concepts of the system, as noted above, the medical device can be configured to receive and store data indicative of past treatment of a person who is the wearer and/or implanted with the medical device. This storage feature can be part of an external component of the implanted device, or could be part of the implantable component, such as where the implantable component includes a memory, such as a silicon-based memory chip or some other device configured to store data, such as store digital data. The memory could be part of the external component of the body worn hearing prostheses. Any device, system, and/or method that can enable the storage of data indicative of past treatment can be utilized in at least some exemplary embodiments.

Note that in an exemplary embodiment, the medical device is configured to receive and store input that is indicative of the past treatment, which input is the data indicative of past treatment noted above. With respect to receipt by the implant, in an exemplary embodiment, this can take a variety of forms, such as for example, a microphone on the body worn device or a microphone that is implanted in the recipient for that matter, where the system can be configured to capture the person's speech and record that data and/or analyze that data and deduce certain features from that data indicative of the past treatment. In one embodiment, the system could record speech after being prompted by the user (a button on the device could be pressed, or a code on the device can be provided to initiate the recordation, or speech recognition to be utilized by the medical device to determine that the following speech should be recorded (the device can be configured to respond to the words “I am inputting treatment information,” for example), or speech recognition can be utilized to determine that what is being said is pertinent to the task of receiving and storing input indicative of past treatment (certain words can connote such). Alternatively, in or in addition to this, the various prompts could be utilized to initiate the analysis of the captured speech. For example, if the recipient stated that “I injected 1 thing of insulin at twelvthirdivife thzaftanoon,” the system could deduce a meaning from this and store something along the following: 1 unit 12:35 p.m., Feb. 21, 2021.

While the embodiment just detailed relies on the user to originate the input, in an alternate embodiment, the medical device could obtain this from another source, such as from a drug delivery dispenser and/or an insulin delivery device (e.g., a delivery patch, a smart syringe, etc.), etc. The delivery device could be in signal communication or otherwise can be configured to provide the data to the medical device (where the delivery device is different/separate from the medical device—the delivery device would also be a medical device). The delivery device could record the data at the time of usage, automatically, or can include data input features akin to that of the medical device just detailed above, the utility being that perhaps it is more convenient for the user to provide input to the delivery device as opposed to the medical device. Still, in a utilitarian embodiment, the delivery device could have a memory or otherwise can be configured to transmit data indicative of the treatment of the person to the medical device. This can be done wirelessly or in a wired manner. This can be done continuously or upon prompting or upon the delivery device being in close proximity to the medical device. Identifiers can be utilized and/or exchanged to ensure that the data is pertinent to the user, etc.

Also, in an alternate embodiment, the medical device could obtain the information based on objective measures. By way of example only and not by way of limitation, biomarkers are utilized in some exemplary embodiments to determine changes in treatment and/or compliance of the recipient with the treatment. As will be detailed below, in some exemplary embodiments, the medical device includes a device that monitors physical phenomenon associated with a human body, such as, for example, features of blood of a human. In an exemplary embodiment, the medical devices herein can identify or otherwise evaluate biomarkers or any other objective measure, and based on the objective measures, deduce the treatment/past treatment/compliance/lack of compliance. In an exemplary embodiment, this can be done automatically by the medical device. In an exemplary embodiment, the obtained data can be transferred to a remote device, in accordance with the teachings detailed herein, where such can be analyzed to determine/deduce the treatment/past treatment/compliance/lack of compliance. In an exemplary embodiment, such can be utilized for the purposes of determining whether or not an intervention is needed or otherwise to determine a treatment going forward. By way of example only and not by way limitation, if there is a lack of compliance with respect to a treatment regimen, it can be deemed less than utilitarian to change the treatment regimen. In an exemplary embodiment, if it has been determined that a patient has not been receiving certain amounts of a therapeutic substance which otherwise should have been received, supplements or emergency provisions of the therapeutic substance can be provided.

In some embodiments, the medical device can be configured to receive text input, such as that which might be the case with respect to an interactive display screen on the medical device. By way of example and not by way of limitation, some medical devices include a digital display, which digital display could be utilized as a touchscreen in some embodiments. The device can be configured to receive a simple code via pushbutton on the device, or by tapping over an implanted microphone or other sensor.

In an exemplary embodiment, the medical device has stored therein data indicative of therapeutic substance(s) taken by the person (which is included in the genus “past treatment”) within at least the past 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 120, 150, 200, 250, or 300 days or more, or any value or range of values therebetween in one day increments. In an exemplary embodiment, data can be temporal data and/or amount data (dosage date, for example) and/or type/brand/name (generic or otherwise).

In an exemplary embodiment, the medical device is configured to communicate some or all of this data regarding the therapeutic substances taken by the person to a second subsystem (treating the medical device as a first subsystem) where the second subsystem can utilize this or a healthcare professional working with the second subsystem can utilize this, to modify or otherwise implement a therapy regimen/treatment regimen.

In some embodiments, the remote handheld device of FIG. 3A could be utilized to receive data upon which the data will be based, and then provide that data to the medical device, which medical device receives and stores such as data indicative of past treatment of the person. Here, the embodiment could utilize the touchscreen or the microphone of the remote handheld device 2401, where the remote handheld device could be in signal communication with another device that has data indicative of the past treatments, and then receive that data, and then conveys that data to the medical device. Corollary to this is that in an exemplary embodiment, this other device could be the device that provides the input to the medical device. For example, the device could be a laptop or desktop computer, or can be a device associated with the household or a building, relating to the Internet of Things. Latent variables can be utilized as well, such as, for example, a GPS or some form of triangulation or location identification device (e.g., what a cell phone utilizes) alone or in combination with a temporal measurement device. In this regard, if the data collection device determines that the person is at a therapy center for over 45 minutes for example, it can be deduced that the person is receiving some form of treatment, and thus this could be provided to the medical device and stored therein (the medical device could do this by itself as well). The data could be qualified as a latent variable and recorded as such so that it could be later analyzed if need be for verification as an actual treatment.

As noted above, in an exemplary embodiment, the system includes a second subsystem. The medical device here can be configured to electronically communicate data indicative of the stored input to the second subsystem. This can be wired or wirelessly executed. The second subsystem could be embodied in the handheld device of FIG. 3, or a laptop or desktop computer, or a specialized device. But in other embodiments, the second subsystem is located very remotely from the medical device and thus the person, and is accessible via a telephone communication system and/or an Internet system, etc. In this regard, in an exemplary embodiment, the aforementioned handheld device and/or laptop computer, etc., could be utilized as the intermediate device to convey the data indicative of the stored input to the second subsystem. This second subsystem can be configured to receive the communicated data and at least one of provide the data in a human understandable format or analyze the data and develop at least a partial treatment regimen based on the analysis.

With respect to the former, this could be accomplished by presenting text on a digital display device, such as a device with a computer monitor (e.g., LCD) or a cathode ray tube, etc. This could also be accomplished in an audio manner. This can also be accomplished utilizing a paper printout for example. In an exemplary embodiment, there is utilitarian value with respect to receiving the communicated data and providing the data in a human understandable format so that a healthcare professional or otherwise a technician can evaluate the data and develop a partial treatment. Some additional scenarios of this will be described below.

With respect to the embodiment where the second subsystem is configured to analyze the data and develop at least a partial treatment regimen based on the analysis (note that the following is also applicable to actions that could be done by a person who has access to the provided data in a human understandable format), the treatment regimen could be an adjustment to the prosthesis or the medical device at issue (e.g., change in a rate or voltage or amperage of electrical stimulation). Alternatively, and/or in addition to this, this could entail the activation of a given feature of the medical device or the deactivation thereof (for example, the activation or deactivation of a body noise cancellation system, the activation/deactivation of a beamforming system—also can be included adjustments thereto). Treatment can also entail obtaining an entirely new medical device. Treatment could be a change in a medication or the amount of medication that should be utilized and/or the frequency/schedule that the medication is taken. Treatment could be the addition of the medication and/or the suspension of another medication. Treatment can also entail obtaining an entirely new medical device. Treatment could also include having the person go to a therapist or to a healthcare specialist or to a technician.

In a utilitarian manner, in an exemplary embodiment, the second subsystem can be configured to communicate the developed at least partial treatment to the medical device and/or to other systems, such as the remote handheld device of FIG. 3A or to an email or to a component of the Internet of things associated with the person.

In some exemplary embodiments, the medical device is configured to receive the communicated developed at least partial treatment and operate in a different manner based on the received communicated developed at least partial treatment. In this regard, the at least partial treatment could be a change in a setting of the medical device or a change in the sensitivity of the medical device with the activation or deactivation of a system of the medical device, etc. This can be done in an automatic fashion. That is, the medical device can be configured to automatically operate in a different manner upon receipt (and in some embodiments, validation) of the communicated developed at least partial treatment.

The above said, in an exemplary embodiment, the device is decidedly not configured to operate automatically in a different manner upon the receipt and/or validation of the communicated developed at least partial treatment. In an exemplary embodiment, the user or a healthcare professional or some other person must affirmatively accept any changes to the operation of the device. In an exemplary embodiment, the user must affirmatively enable the medical device to operate in a different manner. By way of example only and not by way of limitation, in an exemplary embodiment, the medical device could communicate directly to the recipient/person associated with the medical device or could communicate to another component, such as the remote device of FIG. 3A, and convey to the person that it has been deemed utilitarian that the medical device operate in at least a different manner, or otherwise conveyed to the person the proposed change in operation. The person could be asked to accept or reject the change in operation. If the person accepts, then the change can be enabled and the change will happen (either right away or at an opportune/utilitarian time in the future).

Conversely, in an exemplary embodiment, the person must affirmatively make the change to the medical device. That is, any change to the operation must be a change made by a person, such as the person utilizing the medical device. In this regard, in an exemplary embodiment, the medical device could convey information indicating that the change should be made, but the user of the medical device or other pertinent party must make the change.

In view of the above, it can be seen that in an exemplary embodiment, there is no automatic change in the operation of the medical device. In some embodiments, the device(s) are specifically configured to prevent such. In some embodiments, the devices are configured to permit enablement of such, but such is disabled.

In an exemplary embodiment, where for example the medical device is configured to provide tissue stimulation (which includes neural stimulation) to tissue of the person, the medical device is configured to electronically communicate data indicative of the stored data to the second subsystem, as noted above, or to a different system that has the capabilities of the subsystem. Further, the medical device can be configured to receive a communicated developed at least partial treatment developed remotely from the medical device based on the electronically communicated data and operate in a different manner based on the received communicated developed at least partial treatment. Also, the medical device is configured to provide a therapeutic substance to the person in accordance with the received communicated developed at least partial treatment. Here, the second subsystem may not be part of the system, but instead a separate system.

In an exemplary embodiment, the medical device is a glucose monitor, such as a continuous glucose monitor and/or an insulin pump/insulin delivery device, with a monitoring feature. By way of example only and not by way of limitation, FIG. 9 presents the skin facing side isometric view of a blood sugar monitoring device 900 (a continuous glucose monitor), in the form of a patch. This device can instead and/or also be insulin delivery device (as we detail below, utilitarian embodiments utilize separate devices in some embodiments). In this exemplary embodiment, a plurality of transdermal needles 910 are arrayed about the skin facing side of the device supported by a chassis 920. Within the chassis is an electronic logic circuit 930 in signal communication with sensors configured to evaluate samples of body fluids and determine a blood sugar level associated with those samples, or at least determine data indicative of such, which samples can be obtained via one or more of the transdermal needles 910. The logic circuit can also control the delivery of insulin to the recipient of this medical device, in unified monitoring/delivery device 900 (although a separate controller can be used). That said, in an exemplary embodiment, while device 900 includes a logic circuit that can develop a control regimen for the delivery of insulin to the recipient, the device 900 might not be configured to deliver the insulin. Instead, the device 900 could be a master device where a slave delivery device that is physically separate from the device 900 is controlled by the device 900, which slave device delivers the insulin. Accordingly, in an exemplary embodiment, the sensor device is physically separate from the delivery device, and the control of the delivery device can be located in the center device.

Thus, in some embodiments, the system includes a second medical device configured to provide a therapeutic substance to the person, the second medical device being a completely physically separate device from the medical device (e.g., 900 vs. 1005, where 900 can be in signal communication with 1005). Further, the second medical device is configured to receive a communicated developed at least partial treatment based at least in part on the stored data indicative of past treatment and provide the therapeutic substance to the person in accordance with the received communicated developed at least partial treatment.

This control can be based on the aforementioned determined blood sugar level. This logic circuit can vary a treatment regime/delivery regimen of the insulin based on input through an input suite (not shown, but could be wireless or wired via an interface on the opposite side). This logic circuit can have access to a memory that relates to previous blood sugar levels and/or insulin treatment regimens and/or can include logic circuits that can adjust the delivery based on the previous blood sugar level and/or insulin treatment regimens applied to the recipient. This adjustment can be automatic. But alternatively, and/or in addition to this, in an exemplary embodiment, the device 900 can develop a new delivery regimen, and query the recipient/person to accept or otherwise approve the new regimen. Without the approval or acceptance, the new regimen will not be implemented.

Alternatively, in or in addition to this, instead of the medical device developing this new regimen, consistent with the system detailed above, it is the second subsystem (or different system) that develops the new regimen, or a healthcare professional or other person with expertise working with the second system who develops the new regimen. Consistent with the above, this new regimen can be sent back to the user and/or the medical device, where the regimen can be updated. This updating can be automatic, or can instead be manual, if only by approval of the recipient (or the recipient must affirmatively make the adjustments to the device 900 or otherwise changed the controls thereof to implement the new regimen.

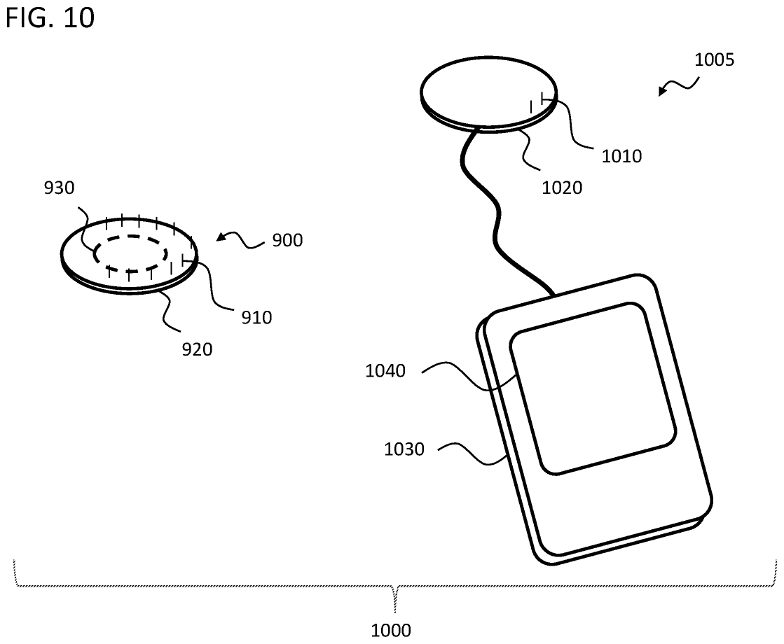

FIG. 10 depicts an exemplary diabetes treatment system 1000. Here, there is the continuous glucose monitor 900 along with the separate insulin delivery device 1005. Here, the delivery piece 1020/the body interface portion with the transdermal needle(s) 1010 is separated from the controller 1030, but in signal communication there with via an electrical cable. In an exemplary embodiment, the controller 1030 also includes an insulin reservoir or otherwise a therapeutic substance reservoir that provides the therapeutic substance, in this case insulin, to the delivery piece 1020. It is noted that in an exemplary embodiment, the delivery piece could be an insulin delivery patch, and the reservoir could be located on/in the patch. A control unit could also be included in the delivery piece, which control unit could be in wired and/or wireless communication with the controller 1030. The controller 1030 could provide control signals to the delivery piece 1020 to implement the insulin delivery regimen. In an exemplary embodiment, a memory could also be included with the delivery piece, which memory could be configured to receive a control regimen from the controller 1030. Here, instead of active one-to-one control where without signal communication between the controller 1030 and the delivery piece 1020, there would be no delivery, upon communication and acceptance of the delivery regimen from the controller 1030, the delivery piece 1020 can operate autonomously, effectively being indirectly controlled by the controller. Put another way, instructions are given to the delivery piece 1020 from the controller 1030, and the delivery piece will operate accordingly until additional instructions/different instructions are given. In an exemplary embodiment, instead of wired communication between the controller and the delivery piece, wireless communication can be utilized. Moreover, in an exemplary embodiment, after control instructions are provided to the delivery piece 1020, the wired connection can be disconnected and remain disconnected until needed at a later date.

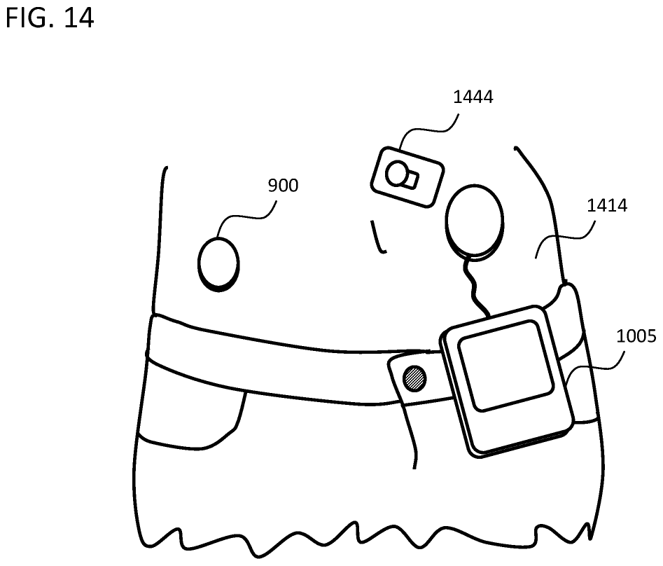

In an exemplary embodiment, controller 1030 could be mounted on a waist belt or on an armband. In this regard, FIG. 14 presents an exemplary scenario use, where the monitoring patch 900 is located on one side of the torso 1414 of a human being, and the drug delivery device 1005 is located on the other side of the torso, where the controller 1030 is clipped to the top of a pair of pants worn by the person.

In an exemplary embodiment, the medical device detailed above is the continuous glucose monitor 900. In an exemplary embodiment, the medical device detailed above is the entire system 1000. In an exemplary embodiment, the medical device is the continuous glucose monitor 900 with the additional feature of being able to deliver insulin from the monitor 900.

Consistent with the teachings detailed above, remote unit 2401 of FIG. 3A for example, could be in signal communication with one or both of the monitor 900 or the delivery device 1005.

In an exemplary embodiment, the medical device detailed above is the system 1000. Thus, in an exemplary embodiment, the controller 1030 can be configured to receive and store input indicative of past treatment of a person who is the wearer of the medical device.

In an exemplary embodiment, the medical device can be configured to electronically communicate data indicative of the stored input to the second system, and the medical device can be configured to provide a therapeutic substance to the person.

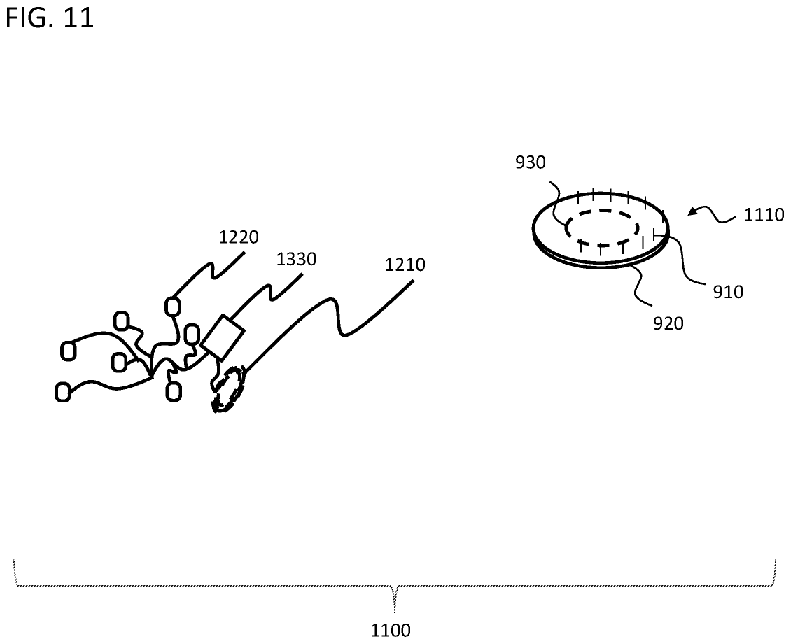

It is briefly noted that in an alternative embodiment of the system the FIG. 10, more accurately, another variation of the system that is somewhat analogous to the system of FIG. 10, device 900 could be replaced with the EEG monitoring devices of FIG. 5-7, and the therapeutic substance delivered by a device 1005 could be a therapeutic substance to treat or manage the effects of epilepsy (e.g., antiseizure medication could be delivered). FIG. 11 thus presents an exemplary epilepsy treatment system 1100, which makes use of the EEG monitoring device of FIG. 6 and a therapeutic substance delivery device 1110 that provides a therapeutic substance to treat epilepsy or otherwise mitigate the effects of epilepsy (e.g., an antiseizure medicine). In this exemplary embodiment, the dispenser 1110 can be similar in principle to the device 900 of FIG. 9, and alternatively, the dispenser 1110 can instead correspond to an apparatus akin to the device 1005 of FIG. 10 (just as it exemplary embodiment, the device 1005 can instead be a device akin to device 1110—all repurposed and/or modified to function in a utilitarian manner for the pertinent systems). Some additional exemplary embodiments of an epilepsy treatment system will be described below.

In view of the above, it can be seen that in at least some exemplary embodiments, the subelements of a given system can be separated. For example, the data processing evaluation/analysis can be separate from the monitoring, and the monitoring can be separate from that and the therapeutic substance delivery and/or the treatment delivery and the therapeutic substance delivery and/or the treatment delivery can be separate from one or both of the data processing or the monitoring. In an exemplary embodiment, one or more of the subelements can be upgraded or otherwise modified without requiring rework of the other subelements. Moreover, failure of one element occurs in a failure mode isolation regimen where such will not physically affect the underlying utility of the other subelements.

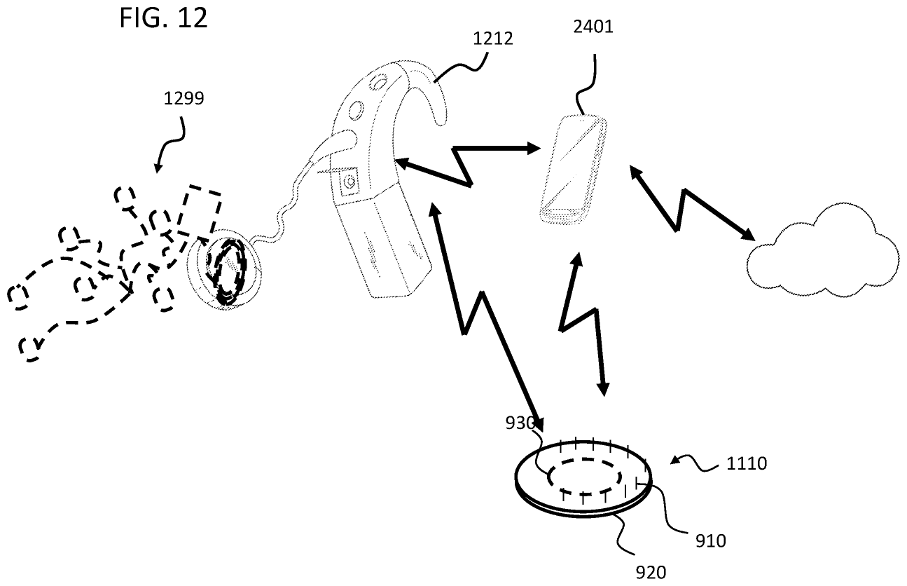

In an exemplary embodiment, there is a closed-loop monitoring and therapeutic substance delivery system, where the closed-loop monitoring and therapeutic substance delivery system is configured to monitor one or more physiological parameters of a user of a medical device. In some embodiments, this system can have access to data indicative of therapeutic substance(s) previously used and/or currently used by the user. In an exemplary embodiment, this can be the system of FIG. 11. In an exemplary embodiment, the housing 1330 can include a memory bank or the like that can store the data indicative of therapeutic substances previously used and/or currently used by the user. While not shown in FIG. 11, system 1100 can further include an external device such as a BTE device 1212 that is in signal communication with coil 1210 via an inductance communication system in a manner analogous to or otherwise the same as that which is the case for a cochlear implant. This is seen in FIG. 12, where device 1299 corresponds to the implanted portion of the embodiment of FIG. 6. Additional details of FIG. 12 will be described momentarily, but here, we note that the BTE device 1212 can include memory components that can store the aforementioned data. Note further that other types of external devices could be utilized, such as an off the ear device that includes an inductance communication coil. Any type of external device usable with the implant that can enable the teachings detailed herein can be utilized in at least some exemplary embodiments. Moreover, device 1299 can be an external component as well. In this regard, the electrodes may not necessarily need to be implanted, or otherwise electrodes that are located on the surface of the skin outside the recipient can be utilized. Of course, a combination of the devices can be utilized—there can be an implanted device that utilizes implanted electrodes, and there could be an external device that utilizes electrodes that lie on the surface of the skin. Both of those devices could communicate with each other and/or with the separate external device which may be in the form of the behind the ear device.

In this exemplary embodiment, device 1110 can correspond to the device that enables the system to be a therapeutic substance delivery system. In an exemplary embodiment, the two components of system 1100 (the implant and/or the eternal device on the one hand, and the delivery device on the other hand) can communicate with each other via a near field RF signal system or any other communication system that can enable communication.

To briefly summarize FIG. 12, FIG. 12 presents a conceptual arrangement where a monitoring device (implanted as shown by representation of the dashed lines, but which can instead or in addition to this be an external device) is configured to directly capture certain data relating to, for example, epilepsy (EEG readings, accelerometer readings (there can be one or more implanted accelerometers and/or one or more accelerometers located in the external component BTE device, or in another component or a plurality of components), audio/sound readings (again, embodiments can utilize a behind-the-ear device 1212, as seen in FIG. 12, in an exemplary embodiment, the underlying behind-the-ear device can be that of a hearing prosthesis or otherwise a modified hearing prostheses behind-the-ear device, and further, combined sensory prostheses can be utilized, such as a hearing implant along with the epilepsy treatment device, and in other embodiments, sound captured from, for example, device 2401 can be utilized, etc.)). In an exemplary embodiment, data based on data obtained by the monitoring device, which can include the raw data, can be provided in an electronic format for example, to the cloud, for example, either directly, or by an intermediary, such as the smart phone 2401 or some other computing device as an intermediary.

That data transmitted over the cloud or whatever transmission regimen that can be utilized, is processed or otherwise analyzed, and in some embodiments, data indicative of a probability of an event that might be deleterious, such as a seizure in this instance, or with respect to diabetes, a diabetic episode, for example, can be derived. Still further by example, recommended dosages and/or changes in dosages of a therapeutic substances and/or new therapeutic substances that can have utilitarian value are identified. Also, in some embodiments, the timing and/or the mode of administering the therapeutic substance can be identified. In some embodiments, utilitarian actions behind simply providing therapeutic substances can be identified, such as, for example, providing an electrical stimulation to the user of the medical device, a determination that it would be utilitarian for the user of the medical device to eat or not eat something, a determination that the user of the medical device should elevate his or her heart rate or decrease his or her heart rate or decrease his or her blood pressure, etc., or even simply increasing a rate of monitoring/sampling. All of this can be done automatically in some instances, such as by utilizing a trained expert system, for example, or some other logic circuit-based device, while in other embodiments, this can be done by a healthcare professional.

In an exemplary embodiment, treatment parameters based on one or more of the above can be developed and transmitted via the cloud or via a phone line or an Internet-based system, etc., to an active component that the medical device user utilizes, such as, for example, a therapeutic substance delivery device either directly or via an intermediate device, again, such as, for example, the smart phone 2401. These components can be completely separate components from the sensing device, or can be part of an overall system, and could be part of an integrated Cingular device, such as the exemplary embodiment of the patch 900 detailed above which is both a monitor and a therapeutic substance delivery apparatus.

Consistent with the embodiments described above, the BTE device 1212, which comprises part of the system configured to monitor one or more physiological parameters of a user of the medical device, here, e.g., EEG, is configured to communicate electronically data based on the data stored therein to a remote device 2401, which can be executed wired or wirelessly. In this exemplary embodiment, remote device 2401 is a smart phone, which can be used communicate to a cloud computing network as depicted, where, for example, via cloud computing, the second system can be accessed. As seen, the communication can be two-way so that the second system can then communicate back to the BTE device 1212. Also shown in the embodiment of FIG. 12 is that the therapeutic substance delivery device 1110 can be in communication with the smart phone 2401 and/or with the BTE device 1212 (or the implant, if the implant can receive signals and/or transmit signals directly to the delivery device—any disclosure herein of using an external device to communicate data from the implant corresponds to an alternate disclosure of using the implant to communicate data, and vice versa, providing that the art enables such). Either way, the delivery device 1110 can obtain data via the cloud and provide data to the cloud.

In an exemplary embodiment, the closed-loop monitoring and therapeutic substance delivery system is further configured to use results from the monitored one or more physiological parameters and, in some embodiments where such data can be accessed/is accessed, the data indicative of therapeutic substance(s) to establish a dosage and/or schedule for future therapeutic substance(s) to be taken by and/or administered to the user. In an exemplary embodiment, this can be executed by, for example, any device that is part of the system, such as, for example, an onboard processor of the BTE device 1212, or an onboard processor of the delivery device 1110. In an exemplary embodiment, a processor per se is not utilized, and instead, lookup tables or the like can be utilized. Note further, the system can utilize computing techniques located at remote location(s) from the sensor portions. For example, a trained expert system at a location remote from the user can be accessed via the cloud or otherwise via the Internet, where the trained expert system can establish the dosage and/or schedule for future therapeutic substances to be taken by and/or administered to the user via their implanted and/or or external device.

Again, as noted above, the system can include a therapeutic substance delivery device to deliver the future therapeutic substance(s). Here, in an embodiment, the system includes a first subsystem, the first subsystem being configured to execute the monitoring and obtaining of the data. This first subsystem can be the device 1299 (and in some embodiments, along with device 1212) or any other of the sensors herein. The aforementioned delivery device can be in signal communication with the first subsystem and/or another subsystem in signal communication with the first subsystem. The delivery device can also be configured to receive data based at least in part on data based on the dosage and/or schedule (data based on data can be the data without variation/modification) or can be a modification of the data (the underlying data). Further, the delivery device can be configured to operate accordingly based on the received data to deliver the future therapeutic substance(s) to the recipient.

In some embodiments, the delivery device is configured to operate to deliver the therapeutic substance(s) according to the dosage and/or schedule only if the recipient approves the operation. By way of example only and not by way of limitation, in an exemplary embodiment, the system can prompt the user for approval of the operation, where, upon sufficient input from the user, the system will operate to deliver the therapeutic substance(s) according to the dosage and/or schedule. The input could be from the user pressing a button or speaking a voice command, where a body worn device or an implanted device could include the input suite to receive the input directly from the recipient. In an alternative embodiment, the input could be through the use of a computer. A response to an email by the user could correspond to acceptance. In an exemplary embodiment, a text message to be utilized, where the recipient accepts the text or the prompt within the text.

That said, in an alternative embodiment, the prompt could come from outside the system, such as by a text or an email. Regardless of how the recipient is prompted, the system will not operate in accordance with the dosage and/or schedule unless the user approves or otherwise puts input into the system. Indeed, there may not be a prompt. Moreover, it is possible that the system can be configured so only the recipient can adjust the system to operate according to the dosage and/or schedule. Put another way, in an exemplary embodiment, the user must affirmatively change the setting, as opposed to simply accepting a change in the setting.

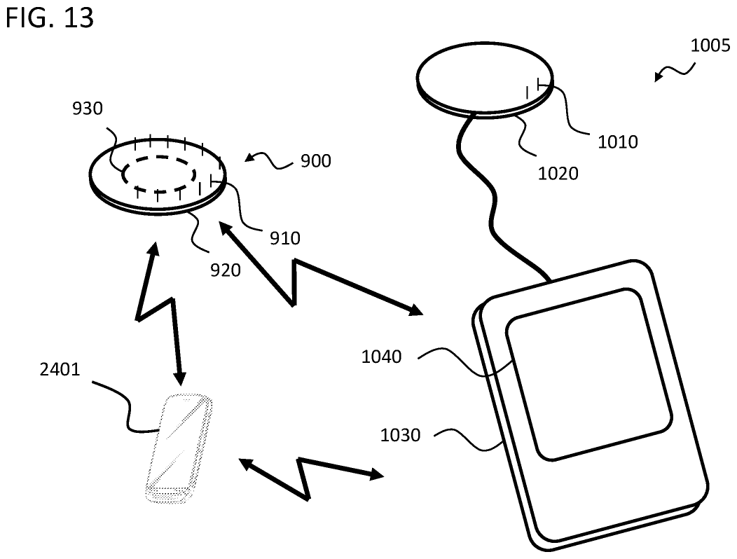

In an exemplary embodiment again where the system includes a first subsystem being configured to execute the monitoring and obtaining of the data, at least the first subsystem or a second subsystem that is a part of the system and is in signal communication with the first subsystem is configured to provide output indicative of the dosage and/or schedule. This output could be provided directly to the user via, for example, a body worn and/or implanted portion of the system. In an exemplary embodiment, such as where the system includes a portion configured to evoke a hearing percept, an artificial hearing percept could be evoked, which would be indicative of the dosage and/or schedule. In an exemplary embodiment, the dosage and/or schedule can be displayed on the display screen 1040 of the device 1105 of FIG. 13 or FIG. 11 by way of example. The dosage and/or schedule can be emailed or texted or provided over a landline for that matter (or via a cellular telephone). In an exemplary embodiment of this exemplary embodiment, the system cannot be changed by itself. It requires the user or some other human to affirmatively make a change. Accordingly, in an exemplary embodiment, the delivery device is configured to enable the user or other healthcare professional to make an adjustment. It is noted that this adjustment could be made remotely. In an exemplary embodiment, a new set of programming or the like could be uploaded by a healthcare professional or some other technician to adjust the operation to achieve the dosage and/or schedule that is been deemed utilitarian.

The above said, in an exemplary embodiment, there is no true change to be made. In this regard, the delivery device can be configured to enable manual initiation of dispensement of the therapeutic substance based on the output. For example, the user will simply operate the delivery device in a manual manner. Without the manual input or initiation by the user, the delivery device will not provide the therapeutic substance according to the dosage and/or schedule.

FIG. 13 presents another exemplary embodiment where the delivery device can be device 1005 as opposed to the patch 1110. Further, the sensor device can be device 900 as opposed to the device 1299. In an exemplary embodiment, the embodiment shown in FIG. 13 can be a diabetes treatment system, consistent with the teachings associated with those components above. Any of the features associated with the components of FIG. 12 can correspond to those of FIG. 13 providing that the art enables such. By way of example only and not by way of limitation, the smart phone 2401 can communicate with the cloud to enable the various features and/or capabilities of the systems just detailed.

Briefly, it is noted that while the sensing and/or monitoring devices relating to the diabetes treatments are typically presented as patches, in alternative embodiments, the arrangement of FIG. 1005 can be utilized as the sensor, and/or a patch to be utilized as the delivery device. And while the embodiments of FIG. 11 and FIG. 12 focus on utilization of a therapeutic substance delivery device 1110 that, in some exemplary embodiments, provides a liquid therapeutic substance, in alternative embodiments, other types of therapeutic devices can be utilized to treat the user, such as, for example, electrical stimulation devices sound therapy, etc. Accordingly, in an exemplary embodiment, an electrode and stimulator assembly can replace the delivery device 1110 by way of example.

In an exemplary embodiment, at least some of the components detailed above can be considered minimally invasive. Such examples are the external devices and the patches detailed above. It is also noted that while various components of the systems detailed above can work in conjunction with each other, in some embodiments, the decidedly are isolated from each other, and in some embodiments, it cannot communicate with each other, at least not directly, and in some embodiments, neither directly nor indirectly, at least not without human intervention.

Embodiments can enable the automated dosage and development and/or adjustment of delivery rates based on physiological data obtained from different devices of the system. For example, with reference to FIG. 12, the delivery rate of the therapeutic substance from the patch 1110 can be based on the data obtained from the sensor device 1299. And as noted above, in some embodiments, there can be an automatic updating/change with respect to the delivery device based on the data from the sensing device, in other embodiments, this is not automatic and the change must be affirmatively made or otherwise at least approved by the user or some other technician.

In view of the ability to sense these physiological features in real time or near real time, or otherwise sensed these physiological features in a utilitarian timeframe, faster changes and/or adjustments and/or developments of therapy regimens can be achieved relative to that which would be the case in the absence of the sensing devices that are implanted and/or worn, etc., by users of the medical device. Moreover, because of the ability to access logged data associated with past treatments and/or past episodes for that matter, in real time or near real time, from the medical device, and owing to the accuracy of such data as compared to that which would be the case if the data was compiled in one fell swoop, or complied at a time frame close to the time that the adjustments were developed and/or made, or otherwise that would be the case if the data was compiled within, for example, a day or two from the time that the adjustments were developed and/or made, in some exemplary embodiments, a dosage level of a therapeutic substance can be varied and otherwise adjusted in a more accurate manner. This can have utilitarian value with respect to enabling the reduction of dosage levels during periods where there is low risk of a deleterious event occurring, such as, for example, an epileptic seizure. By way of example only and not by way of limitation, data that can be obtained from the sensors, even in real time or near real time, may not necessarily tell the full story. It is the past history, in combination with the current state of the user of the medical device, that can be used to better estimate or otherwise forecast or otherwise determine the future state of the person using the medical device.

Corollary to this is the ability of the embodiments herein, at least some of them, to support the delivery of fast acting treatment(s) with the same delivery system supporting continuous treatment or, via a standalone delivery system. By way of example only and not by way of limitation, in an exemplary embodiment, the delivery device 1005 can have two or more reservoirs located therein; one for the continuous delivery, and one for a special delivery or otherwise an aberrant deliver or an emergency delivery. The therapeutic substances within the reservoirs could be different. Upon a determination that the recipient is potentially going to experience an event that could be deleterious, or a determination is made that there is an increased probability that such might be the case, which event might want to be prevented or otherwise mitigated, the therapeutic substance from the “special” reservoir could be utilized for the substance that will be delivered to the person using the medical device, in lieu of and/or in addition to the continuously delivered substance. Alternatively, in an exemplary embodiment, a separate delivery device could be utilized in this regard, by way of example only and not by way of limitation, such as another delivery device already operationally attached to the recipient but separate from the patch 1110 and/or the delivery device 1005, such as fast delivery bolus 1444 of FIG. 14, could be activated (this could be in signal communication with the control unit, or another device, or the phone, etc.), to provide this different therapeutic substance. Still further, the recipient/user could be prompted automatedly by the system of which the medical device is apart, including, for example, by the medical device that is attached to the recipient and/or implanted in the recipient, and/or prompted by a healthcare professional, to utilize another drug delivery device, not necessarily worn or otherwise attached to the recipient, that the recipient/user maintains in close proximity to himself or herself. By way of example only and not by way of limitation, this can be a syringe that is preloaded with a therapeutic substance, or, the recipient could load the syringe himself or herself and then utilizes syringe. Note also that consistent with the teachings detailed herein, in at least some of these embodiments, the delivery could be automatic, while in other embodiments, the delivery could require the affirmative acceptance of the recipient, while in other embodiments, the delivery could require the recipient to actually initiate the delivery (e.g., by activating the secondary delivery device with a tap code, or by placing a magnetic activator next to the secondary device to cause the secondary device to deliver therapeutic substance, or by otherwise turning that secondary delivery device on—in an exemplary embodiment, a force of sufficient magnitude onto the outward facing surface of bolus 1444 could inject a pre-positioned needle into the skin and provide the secondary therapeutic substance). Corollary to this is that in some embodiments, the delivery of the therapeutic substance can be faulted or otherwise suspended, if it is determined that continued use of the therapeutic substance could be causing undesirable effects.

In an exemplary embodiment, the fast acting device could have a small needle positioned in the dermis of the skin (which could be utilitarian with respect to being a viable option for delivery of AEDs). The fast acting device that includes a reservoir, which it held one or more bolus drug treatments that could respond to the prediction of a probability of a deleterious event.

While the embodiment of FIG. 14 presents the fast acting drug delivery device 1444 located on the torso 1414, in an alternate embodiment and/or in addition to this, it can be located elsewhere, such as, for example, when the side and/or the back of the upper arm, or lower arm, etc. Indeed, with respect to embodiments where the needle has not already pierced the skin, the needle could be retracted into a wristwatch like device or an armband like device, where the user could take his or her other arm and turns a dial face of the like which drives the needle into the skin, converting the rotational movement to a vector movement, or otherwise releases a spring-loaded device that drives the needle into the skin, where thereafter, the fast acting drug is delivered. This all can be performed automatically as well. Upon receiving a signal that the fast acting drug is needed, an on board battery of this device can be utilized to drive the needle into the skin or otherwise actuate a release with a spring-loaded component provides the force.

Thus, as can be seen, embodiments can use objective measures, such as where a person's epilepsy profile is quantified to adjust the continuous drug delivery and/or support the delivery of a fast acting agent either manually or by automated algorithms, etc.

While the embodiments described above focused on separate different therapeutic substances, in an alternative embodiment, instead of and/or in addition to this, an amount and/or rate of the therapeutic substance already being delivered could be increased or decreased on a real-time basis to address the forecast or otherwise treat this sensed change in condition. That is, the continuous dosing could be modulated based on the probabilities and/or based on the analysis of the data based on the sensed data, along with an evaluation of the past history of the user of the medical device.

Moreover, embodiments include scenarios where the therapeutic substance associated with the medical devices that are attached to the recipient are not being delivered on a continuous or scheduled basis. Instead, they are to be delivered only on an as-needed basis. Accordingly, the teachings detailed herein can be utilized to determine that there is a need for such, whereupon the therapeutic substance can be delivered, automatically, or via the cooperation of the user.

With respect to some embodiments, the amounts and/or types of therapeutic substances can be better controlled or otherwise utilitarianly controlled in a more limited temporal manner relative to that which would otherwise be the case with respect to the teachings detailed herein. This can have utilitarian effect of reducing side effects and cost savings, because the therapeutic substance could be utilized only when needed as opposed to being utilized in an abundance of caution scenario.

Embodiments herein can have utilitarian value with respect to improving compliance with respect to individuals taking drug treatments. This can be because the teachings detailed herein provide a centralized data collection method and/or a centralized data storage method, which data collection and/or storage method can stay with the recipient.

Moreover, in view of the fact that at least some exemplary embodiments of the teachings detailed herein have access to the past history of the user of the medical device, the past history can be utilized for comparison purposes to other like members of a given population, which can be utilized to alter a treatment or otherwise develop a new treatment regimen and/or otherwise be used to identify the occurrence of a possible deleterious event. This can be done by utilizing a trained expert system and/or a machine learning system and/or an artificial intelligence system. In this regard, the second system detailed above can be such a system. Owing to the ability of at least some embodiments to communicate over wide distances with a remote system or a remote healthcare professional, recently identified characteristics associated with a statistically similar population to which a user of the medical device potentially belongs can be relatively quickly applied for comparison purposes to the user of the medical device, and therapy regimes can be developed based thereon (or indicators indicative of an oncoming deleterious event can be relied upon from that statistically significant population), again relatively quickly, which therapy regimes have been shown to have a statistically utilitarian result with respect to that population.

Some exemplary methods and scenarios of use will now be described. In some embodiments, there is no affirmative physician and/or registered nurse and/or nurse practitioner adjustment of dosages and/or drug selection of the devices and systems detailed herein. In an exemplary embodiment, it is always a user or other technician or other healthcare professional that executes such actions and/or one or more of the actions detailed herein. In an exemplary embodiment, there is no prescription developed by a physician and/or registered nurse and/or nurse practitioner or otherwise by a person licensed in the United States of America to prescribed such.

Further, in some exemplary embodiments, there is no self-reporting or otherwise subjective feedback from an individual in real time when adjustments to dosages and/or drug selection and/or the various changes to the operation of the device are developed. That is, for example, any data upon which the adjustments to the dosages and/or drug selections and/or other changes are based upon coming from the data associated with the medical device (it is not considered self-reporting if the person inputted this information in the medical device in accordance with the teachings detailed herein, whether medical device stores the data for later usage. In an exemplary embodiment, the time period from when the user inputs at least some data and/or most of the data and/or all of the data into the medical device or other systems detailed herein to the time that the data from the medical device is utilized as a basis to make or develop adjustments and/or change a therapy regimen and/or change or identify drug selection and/or execute one or more of the actions detailed herein, is at least 1, 2, 3, 4, 5, 6, 7, 8, 9, 10, 15, 20, 25, 30, 35, 40, 45, 50, 55, 60, 70, 80, 90, 100, 110, or 120 days or more, or any value or range of values therebetween in 1 day increments.

With respect to an exemplary embodiment associated with epilepsy, again, there is little to no reliance on self-reporting of seizures, in some embodiments.

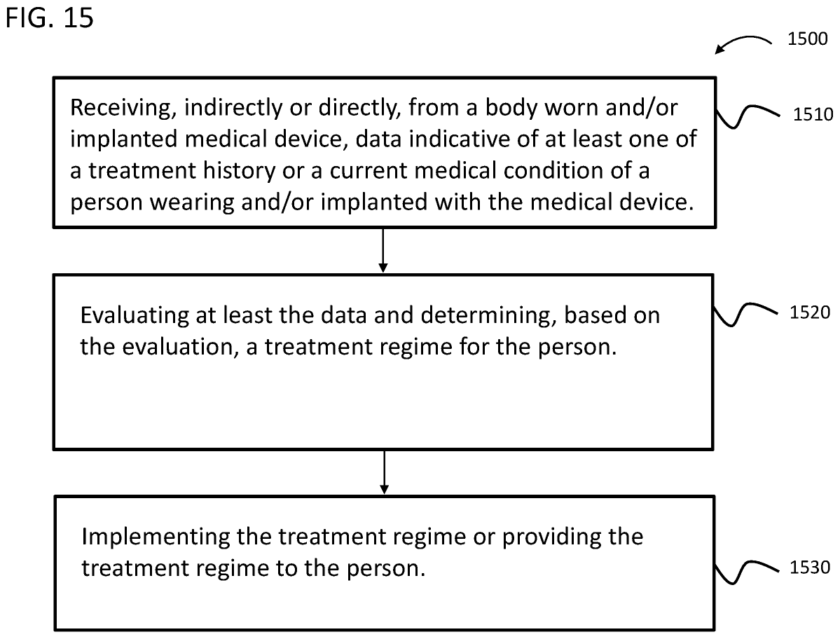

FIG. 15 presents an exemplary algorithm for an exemplary method, method 1500, which includes method action 1510, which corresponds to the action of receiving, directly or indirectly (e.g., via an intermediary, such as the smart phone 2401) from a body worn and/or implanted medical device, such as the device 1299 of FIG. 12 (which would be indirect receipt), or from device 1212 (which would be direct receipt), data indicative of at least one of a treatment history (number of injections of a drug or number of pills of a drug taken, amounts of a drug temporal data associated with the usage of such drugs, etc.), or a current medical condition (e.g., EEG data indicating a seizure has increased probability of occurring, difficulty in breathing, a blood sugar level etc.) of a person wearing and/or implanted with the medical device. The data can be based on or otherwise be obtained from the various devices detailed herein. The data can be received over a computer in a cloud computing regimen or via other data transmission regimes, such as via an email packet, etc.

Method 1500 further includes method action 1520, which includes the action of evaluating at least the data that is received and determining, based on the evaluation, a treatment regimen for the person. As noted above, this can be executed in an automated fashion, and thus in an exemplary embodiment, an expert system that is trained can be utilized to execute method action 1520.

Corollary to this is that in an exemplary embodiment, there can be a computer readable code for executing method action 1510 and computer readable code for executing method action 1520.

Method 1500 further includes method action 1530, which includes the action of implementing the treatment regimen or providing the treatment regimen to the person. In an exemplary embodiment, with respect to the former, this can be executed by a healthcare professional or other technician taking control, albeit temporarily, of the medical device and causing the medical device to operate in a manner concomitant with the treatment regimen. In an exemplary embodiment, this can be executed by uploading a program to cause the medical device to operate according to the treatment regimen. Still further, in an exemplary embodiment, this can be executed by adjusting a setting for example, or implementing a routine, of the medical device. In an exemplary embodiment, there is computer readable code for executing method action 1530, at least in some exemplary embodiments.

In an exemplary embodiment, the body worn and/or implanted medical device is configured to sense a physiological phenomenon of the person, and at least prior to the action of receiving (e.g., 1, 2, 3, 4, 5, 6, 7, 8, 9, 10, 11, 12, 13, 14, 15, 20, 25, 30, 35, 40, 45, 50, 55, or 60 days or more, or any value or range of values therebetween in 1 day increments), has stored therein the data indicative of the treatment history of the person. In this exemplary method, the person is completely conscious, and the action of evaluating is executed without real time input from the person regarding the treatment history.

In an exemplary embodiment, the body worn and/or implanted medical device is configured to sense a physiological phenomenon of the person, and at least prior to the action of receiving, has stored therein the data indicative of the treatment history of the person, and the action of evaluating and determining is executed automatically using artificial logic (as opposed to natural logic of a person). In an exemplary embodiment, the artificial logic is embodied in an artificial intelligence device, while in other embodiments, it is a more traditional computing device. In an exemplary embodiment, the artificial logic is a trained expert system.

In an exemplary embodiment, method 1500 further includes the action of implementing the treatment regimen automatically (as opposed to human implementation, such as a human utilizing a syringe to administer a therapeutic substance). Further, the person has a therapeutic substance delivery system attached thereto and the action of implementing the treatment regimen automatically causes the therapeutic substance delivery system to automatically provide the therapeutic substance in a quantity and/or quality automatically determined in the action of determining the treatment regimen. In some embodiments, consistent with the teachings detailed above, the therapeutic substance delivery system is part of a second medical device physically separate from the medical device. By way of example only and not by way of limitation, the second medical device can be a manual syringe and/or an automated intravenous delivery system. In an exemplary embodiment, a scenario of use of a syringe, manual or otherwise, can be an emergency situation, where the user of the syringe may be under duress (the user could be the person who is to be injected). In an exemplary embodiment, the user (recipient or caregiver) operating the delivery of drug (here, a syringe, but can be one or more of the other medicament delivery devices detailed herein, and others) is doing so based on feedback from the system of which the medical device is apart, including the medical device, indicating one or more of when, how long, and how much of the drug is to be delivered. In an exemplary embodiment, the second device can be a smart syringe, where the system instructs and/or provides data to the smart syringe, and the smart syringe automatically meters/sets the dosage to be delivered. In other embodiments, the therapeutic substance delivery system is an integral part of the medical device.

Consistent with the focus of at least some exemplary embodiments, in an exemplary embodiment of method 1500, the person wearing and/or implanted with the medical device has epilepsy. In an exemplary embodiment, the person is experiencing an epileptic seizure, while in other embodiments, the person may be about to experience an epileptic seizure. Further, in an exemplary embodiment, the treatment regimen is at least in part administration of a fast acting anti-seizure and/or seizure management therapeutic substance, which could be a drug or other medication, and, consistent with embodiments detailed above, the therapeutic substance can be delivered transdermally. In an exemplary embodiment, a quantity and/or quality of the therapeutic substance that is or will be administered is based at least in part on the evaluation. This can be implemented by adjusting a feature of the continuous delivery regimen. This can be implemented by determining how much of the fast acting therapeutic substance should be delivered (e.g., in a severe case, more might be delivered relative to that which would otherwise be the case in a less severe instance, the quantity might be varied depending on whether or not the continuously provided substance will continue to be provided, there could be utilitarian value with respect to breaking up the administration of the fast acting drug, and thus, for example, 70% of a total desired fast acting therapeutic substance can be delivered during a first time period, and the remaining 30% might be delivered later). With respect to quality, different types of therapeutic drugs can be utilized, again depending on the severity of the situation.

In an exemplary scenario of method 1500, the person is experiencing a deleterious medical episode having begun within or at 0.1, 0.2, 0.3, 04, 0.5, 0.6, 0.7, 0.8, 0.9, 1, 2, 3, 4, 5, 6, 7, 8, 9, 10, 15, 20, 25, 30, 35, or 40 hours, or any values or range of values therebetween in 0.1 hour increments (e.g., 9.3 to 22.2, 10.3, 1.2, etc.) relative to the action of receiving in method action 1510. In an exemplary embodiment, consistent with the aforementioned scenario just described, the treatment regimen is at least in part administration of a fast acting therapeutic substance, and a quantity and/or quality of the medication that is or will be administered is based at least in part on the evaluation.

In an exemplary embodiment, the person who is wearing and/or has been implanted with the medical device has been taking a therapeutic substance to treat an ailment for at least and/or equal to 3, 4, 5, 6, 7, 8, 9, 10, 15, 20, 25, 30, 35, 40, 45, 50, 60, 70, 80, 90, 100, 120, or 150 days or more, or any values or range of values therebetween in 1 day increments, and prior to the action of determining in method 1500, the action of evaluating and determining results in a treatment regimen for the person that results in a decrease in an average daily amount of the therapeutic substance during the time of implementing the treatment regimen.

In an exemplary embodiment, the body worn and/or implanted medical device is configured to sense a physiological phenomenon of the person. This can be the EEG measurements, or could be the blood sugar measurements, or any other of the physiological phenomenon as disclosed herein others. Method 1500 further includes implementing the treatment regimen automatically. In some embodiments, the person has a therapeutic substance delivery system attached thereto and the action of implementing the treatment regimen automatically causes the therapeutic substance delivery system to automatically provide a fast acting therapeutic substance in a quantity and/or quality automatically determined in the action of determining the treatment regimen. In some embodiments, for at least 3, 4, 5, 6, 7, 8, 9, 10, 15, 20, 25, 30, 35, 40, 45, 50, 60, 70, 80, 90, 100, 120, or 150 days or more, or any values or range of values therebetween in 1 day increments prior to the action of implementing the treatment regimen, the person has been receiving therapeutic substance in amounts and/or types substantially different from that provided during the action of automatically providing. This can be a therapeutic substance that is continuously delivered and is different from the fast acting therapeutic substance. Alternatively, this could be a substance that has been continuously delivered to the recipient what is now being delivered in higher (or lower) dosages.

In an exemplary embodiment, the amounts of a given therapeutic substance that is delivered is at least 10, 20, 30, 40, 50, 60, 70, 80, 90, 100, 125, 150, 175, 200, 250, 300, 350, 400, 450, 500, 600, 700, 800, 900 or 1000 percent or any value or range of values therebetween in 1% increments greater than the amount that was previously given, such as the continuously given amount.

In an exemplary embodiment, in addition to the method actions detailed above with respect to method 1500, there can be the additional action of receiving additional data from the medical device, the additional data indicative of current physiological information about the person. The method can further include the additional action of varying the quantity and/or quality of the therapeutic substance during administration based on the received additional data. Thus, it can be seen that in an exemplary embodiment, the patient can be monitored during an event, such as being monitored in real time, and this monitoring can be done remotely, and adjustments to the therapy can be made in real time and/or in near real time. In this regard, in an exemplary embodiment, the action of receiving additional data can occur within or at 0.1, 0.2, 0.3, 04, 0.5, 0.6, 0.7, 0.8, 0.9, 1, 2, 3, 4, 5, 6, 7, 8, 9, 10, 15, 20, 25, 30, 35, or 40 hours, or any values or range of values therebetween in 0.1 hour increments (e.g., 9.3 to 22.2, 10.3, 1.2, etc.) relative to the action of implementing the treatment regimen. In an exemplary embodiment, the offer mentioned time periods are relative to the action of administration of the fast acting therapeutic substance.

Again, not all embodiments are directed towards the treatment of epilepsy. In an exemplary embodiment, the medical device that is the subject of method 1500 is a hearing prosthesis. In an exemplary embodiment, the treatment regimen could be an adjustment to that hearing prosthesis, such as the stimulation rate and/or the voltage and/or amperage that is applied by a cochlear implant. In an exemplary embodiment, the adjustment could be an adjustment to the threshold level and/or comfort level. Still further, consistent with the embodiments where the hearing prosthesis can be configured to provide stimulation and also sense a physical phenomenon associated with the recipient, in an exemplary embodiment, the hearing prosthesis was utilized in conjunction with the person to evaluate the person's hearing, and the data that is received in method action 1510 is indicative of a current medical condition, wherein the current medical condition is the person's ability to hear, and the data is based on data obtained when using the hearing prosthesis to evaluate the person's hearing.

In view of the above, it can be seen that there is utilitarian value with respect to repeatedly testing hearing of a person who suffers from a hearing disorder, over the course of months and/or years, and based on a determination that a person's hearing has changed, providing a treatment regimen that corresponds to the adjustment and/or the addition of a therapeutic substance to treat hearing loss/a hearing disorder related ailments. Note that the therapeutic substance provided to the recipient need not necessarily treat hearing loss per se. For example, it is possible that infections or the like can result from a hearing prosthesis. Inflammation can exist. A recipient of a hearing prosthesis' ability to hear or otherwise perceive sound can serve as a latent variable for the potential of an infection or inflammation or otherwise that there is a reaction to the hearing prostheses. Thus, in an exemplary embodiment, anti-inflammatory and/or anti-infection therapeutic substances can be provided.

It is noted that a conventional hearing aid is a hearing prosthesis as used in the specification. Accordingly, embodiments can be directed to utilizing a conventional hearing aid to periodically test a person's hearing, wherein the data of a current medical condition of a person wearing the medical device is the ability of the person to hear. A treatment regimen can then be developed for that person if it is determined that the ability of the person who here has changed.

It is briefly noted that while most embodiments herein are directed towards a deleterious event occurring or otherwise the further degradation of a feature of a human (e.g. hearing), embodiments could exist where the current medical condition corresponds to an improvement in the person. Accordingly, a treatment regimen that is implemented according to method 1500 could be the implementation of a relaxation of a prior treatment. For example, embodiments where the current medical condition is the absence of tinnitus where previously the person had tinnitus could result in a treatment regimen that relaxes the tinnitus treatment or otherwise in fact eliminates the prior tinnitus treatment.

Note also that embodiments can relate to the ear system in general but not the hearing related. By way of example only and not by way of limitation, embodiments can be directed towards management or treatment of vestibular balance disorders. The data could be related to a current vestibular disorder, and the treatment regimen could be a therapeutic substance retreating such.

In view of the above, it can be seen that in at least some exemplary embodiments, the physiological parameters that are monitored and/or actions associated with determining the current medical condition of a person wearing and/or implanted with the medical device can have been going on for days or weeks or months or even years before the action of implementing a treatment regimen, or even any of the actions associated with method 1500. Accordingly, in an exemplary embodiment of method 1500, method 1500 further includes the action of receiving the data over the course of at least 1, 2, 3, 4, 5, 6, 7, 8, 9, 10, 11, 12, 13, 14, 15, 16, 17, 18, 19, 20, 21, 22, 23, or 24 or more months, or any value or any range of values therebetween in one day increments.

In an exemplary embodiment, method 1500 can be executed at least in part (or in whole in some embodiments) in an emergency room and/or by paramedics operating from an ambulance (in an exemplary embodiment, method 1500 can be executed in an ambulance or in a residential location by paramedics). By way of example, the emergency room (or ambulance) can be configured to receive data from the body worn and/or implanted medical device, or otherwise include tools or equipment that are configured to do so. This can be achieved via coordination between manufacturers of the medical device and the equipment in the emergency room or otherwise tools available within an emergency room. If an emergency room is able to obtain readings directly from and/or in real time that are taken from the medical device, the time to commence or otherwise begin treatment can be reduced relative to that which would otherwise be the case. With respect to an embodiment where the entire method is executed in the emergency room, the equipment in the emergency room and/or the implant can be precertified so that the emergency room or ambulatory healthcare professionals can rely upon the data from the medical device.

In this regard, in an exemplary embodiment, the implementation of the treatment regimen is executed based totally on the data from the body worn and/or implanted medical device. This can be done owing to the precertification and the relatively high confidence that the medical device is providing data that is accurate and otherwise utilitarian.

Accordingly, in an exemplary embodiment of method 1500, there is a person who is experiencing an epileptic seizure, and this person is in an emergency room of the hospital, or have arrived at the location where the person is located by ambulance. The healthcare professionals in the emergency room/having arrived by ambulance, receive the data utilizing their equipment which is configured to receive the data from the medical device used by the patient. They can evaluate the data and determine a treatment regimen for the person, or they can utilize a machine to do so. Note also that in an exemplary embodiment, this evaluation in the termination could potentially be done prior to the healthcare professionals having direct access to the person. This can be achieved via a pre-arranged system with secure data transfer and data acquisition componentry to ensure that the data that the emergency healthcare professionals are utilizing is accurate and can be relied upon for the purposes of emergency medicine. In this regard, in an exemplary embodiment of the implementation of method 1500, the equipment that is utilized by the emergency healthcare professionals to evaluate the data and determine based on the evaluation, the treatment regimen, is equipment that meets the approval of the FDA for standard use in such situations.

Thus, it can be seen that in an exemplary embodiment, the emergency healthcare professionals are relieved from the action of applying sensors to a person in distress and/or waiting for those sensors to sense the physiological features of the person of interest. In this regard, in an exemplary embodiment, method 1500 is executed without applying sensors to the person and/or without waiting for those sensors to operate as they already have been applied and they have already operated. Along these lines, the sensors can have been applied over 15, 30, 45, 60, 90, 120, 200, 300, 400, 500, or 600 days or more or any value or range of values therebetween in one day increments from the action of receiving of method 1500.

Method 1500 can have utilitarian value with respect to adjusting the implemented treatment regimen while the treatment regimen is being implemented. By way of example, the sensors of the medical device can sense in real time the physiological features of the person in distress, and that medical device can be relied upon by the emergency healthcare professionals to provide data in real time regarding the condition of the person, and thus can utilize that data to adjust a treatment. For example, in the case of an epileptic seizure, a rate and/or an amount of the therapeutic substance being administered might be changed or otherwise varied during the treatment owing to changed physiological conditions of the recipient. These changed physiological conditions of the recipient can be determined utilizing the data from the medical device in real time or near real time.

Moreover, the ability to have the physiological features more quickly than that which would otherwise be the case in the absence of the medical device that is worn or implanted into the person can enable faster identification of the underlying therapeutic substance that is utilitarian to provide to the recipient in the first instance. That is, the emergency healthcare professionals can utilize the data from the medical device to determine what type of therapeutic substance should be given, if any for that matter, to the person under distress. Again, in some embodiments, this decision can be done without utilizing any non-implanted and/or body worn sensors. That is, the decision can be made based totally on the data from the medical device. The above said, in an exemplary embodiment, there could be limited usage of other sensory devices, such as for example a stethoscope and/or an eye scope and/or a blood pressure monitor device. Thus, in some embodiments, there would be no duplication of the type of sensor that is part of the medical device (for example, EEG data would be totally from the medical device, and not from a separate device that is in the long-term possession of the emergency healthcare personnel), but there could be other devices that would be applied as a general precaution or otherwise in the standard course of any general evaluation of a person. In some embodiments, at least there would be no duplication initially. During the course of the treatment, there might be duplication and/or there might be other types of tests run with other types of sensors. The key here is that the initial treatment can be begun based totally on the data from the body worn and/or implanted medical device.

Moreover, in some exemplary embodiments, there is the action of titrating the dosage of the therapeutic substance while injecting the therapeutic substance into the person.

Corollary to the above is the ability to have a feedback loop that exists in real time while the person is being treated, which feedback loop relies on the body worn and/or implanted medical device. This feedback loop can be utilized to adjust the implemented treatment regimen in real time during the treatment process. In at least some exemplary embodiments, the feedback loop and/or the treatment regimen is stored in the medical device and/or at a remote location. It is noted that in at least some exemplary embodiments, treatments/actions that are unsuccessful or otherwise deemed less utilitarian than others (e.g., in the instance where, for example, there are side effects that are not wanted side effects that are deemed unacceptable) are also stored or otherwise recorded, in the device and/or at a remote location, so that such can be analyzed or otherwise so that the treatment regimen can be avoided or otherwise excluded in the future, such as in embodiments where, for example, the treatments are adaptive or otherwise are revised at a later date.

While the embodiment just under discussion has been directed towards the concept of treating an epileptic seizure in real time, embodiments also include predicting the possibility of a future occurrence of a seizure, as noted above. In an exemplary embodiment, the utilization of the past treatment history serves as a historical profile of the person who is wearing or otherwise is implanted with the medical device. Different people who suffer from epilepsy will react differently to the same type of drug. Corollary to this is that different people will have a same reaction to a drug that is provided in a different manner. For example, some people will react more utilitarian to an initially quick dose of a therapeutic substance than others, and vice versa. In this regard, the timing of the delivery of quantities of a therapeutic substance will have different utility for different people. Accordingly, the treatment history that is stored in a medical device that is accessible to healthcare personnel or the like can be—will be—specific to that person. The things that work and the things that do not work and the things that work better than others will thus be known in at least some embodiments. Accordingly, the techniques detailed herein with respect to evaluating the data that is stored in the body worn and/or implanted medical device and determining a treatment regimen for that person take into account the historical profile of that person and will thus, in at least some embodiments, be influenced by the techniques that work better than other techniques. Thus, embodiments of developing the treatment regimen will include developing treatment regimes based on past successful and/or unsuccessful treatments of the person.

In an exemplary embodiment, the body worn and/or implanted medical device represents the most up-to-date set of data associated with the person's treatment history.

Below is presented a chart detailing some exemplary features of some exemplary embodiments, where the embodiments relate to epilepsy and/or deep brain and/or vagal nerve stimulation and/or monitoring. Below, if a drug is mentioned, it is noted that in an alternate embodiment, treatment can instead or otherwise be augmented with neurostimulation, such as by way of example, of the vagal nerve and/or the brain in an effort to minimise the incidence and/or prevalence of epileptic seizures. Below, the data stored and/or exchanged corresponds to the data that is stored in the medical device with the system associated there with as detailed above. The origin corresponds to how the data was obtained. As seen below, sensors are mentioned, and this is concomitant with the embodiments that utilize a monitoring device. Note also that below, subjective origins are detailed. This is concomitant with the embodiments that enable a recipient to input into the device or to a system associated with the device data that can be used according to the teachings detailed herein. To be clear, in at least some exemplary embodiments of the devices and systems and/or methods herein, the person of interest with the person who utilizes the medical device can input subjective data to the system, which data can be used to implement the overall teachings detailed herein.

| Data Stored | | | |
|---|---|---|---|
| and/or | | | |
| Exchanged | Origin | Some exemplary scenarios | Exemplary Application |
| Brain activity | sensors | monitoring of | real-time monitoring of a |
| (EEG), heart | | baseline brain | drug administration |
| (ECG), | | activity and events | during a status epileptics |
| respiration and | | (such as seizures, | event in the hospital |
| blood oxygen | | sub-clinical seizures | monitoring of the drug |
| saturation | | and inter-ictal | efficacy of the drug |
| (SpO2) | | spiking) | treatment (using the |
| | | monitor the vital | inter-ictal activity of the |
| | | signs of the patient | EEG as a biomarker). This |
| | | and detect any | data is then fed back to |
| | | abnormalities | the cloud or the drug |
| | | around respiration, | delivery device (or |
| | | cardiac function etc. | treating physician) |
| | | | via the vital sign |
| | | | monitoring provide |
| | | | objective assessment of |
| | | | drug side effects that |
| | | | may cause changes in the |
| | | | vital signs, e.g., possible |
| | | | overdosing |
| historical of | memory + | The implant stores the | To assess if drug treatment is |
| drug use and | sensor | historical patient | currently effective at slowing or |
| efficacy | | information in an effort to | improving seizure minimisation. |
| | | exchange this to optimise | To advise the physician to adjust |
| | | the treatment of the | or modify the dose |
| | | individual | |
| patient | subjective | Patient reported | This information can be utilised |
| reported | | information relating to their | to exchange with the treating |
| information | | own assessment of either | physician (along with the |
| | | their well-being, seizure | sensors) to ‘close the loop’ on |
| | | activity and cognitive | the drug treatment and whether |
| | | function. | the prescription needs to |
| | | | change. |

Embodiments are also directed towards the utilization of at least parts of a medical device across multiple users. In an exemplary embodiment, there are therapeutic substance delivery systems that can be employed on a number of individuals in a manner that is more utilitarian than that which currently exist using present technologies. In this regard, current technologies presented the situation whereby having a plurality of users associated with the same delivery system, or at least a portion of the delivery system, there could be complications with respect to ensuring the appropriate drug or dosage is prescribed for the individual interacting with the system.

Another scenario that exists with the current commercial use of current medical devices is that the amount of drug dosage and timing of administration is controlled solely by the user or physician. This leads to the treatment being entirely prescriptive in nature in that the dosage is predetermined by the physician and does not take into account the individual's current disease/condition state (e.g., epilepsy and risk of seizure). At least some exemplary embodiments detailed herein can remedy this or otherwise expand upon this limitation.

Embodiments can include a wireless (in some embodiments, it is wired) inter-connection between an implanted or wearable technology in/on the user and the therapeutic substance delivery system. In some exemplary embodiments, the medical device in/on the user is the master device and the drug delivery device is the slave device in that the latter will respond according to the wireless (or wired) exchange of information the delivery device receives from the user's medical device. In some embodiments, the drug delivery device is configured to respond to the master device's request to adjust one or more of dosage of the therapeutic substance, or selection of the substance, timing of administration of the substance. (In an exemplary embodiment, based on either the physician's prescribed recommendation for drug administration or based on the master device's input on utilitarian timing based on the patient's current disease state or condition.)

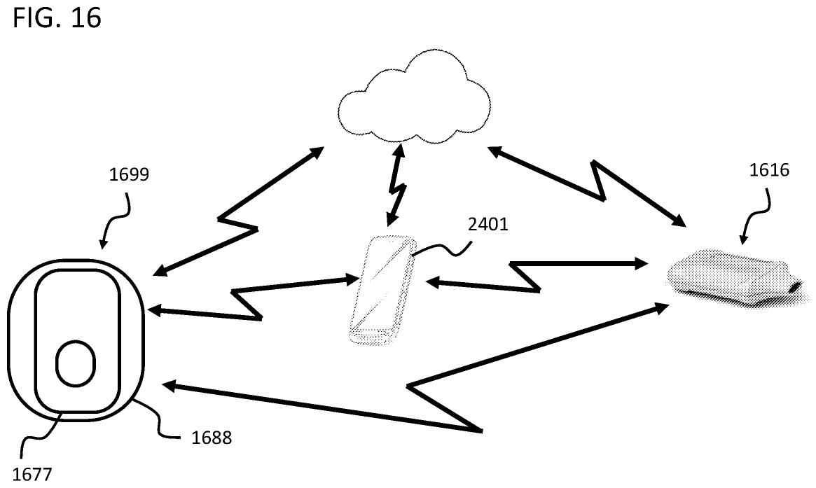

FIG. 16 presents an exemplary embodiment that can be utilized to implement the just described innovative features. Here, device 1699 is an external medical device that can be utilized to sense phenomenon associated with the recipient. In this exemplary embodiment, device 1699 includes an accelerometer. Device 1699 can include an adhesive pad 1688 on which is located in housing 1677, in which the housing includes the accelerometer and receiver and/or transmitter and/or transceiver componentry. Also included in the housing can be a logic circuit that is configured to evaluate the outputs of the accelerometer and evaluate such and determine whether or not there is utilitarian value with respect to transmitting the data for further analysis. That said, the device 6099 can simply continuously or periodically transmit data based on the output of the accelerometer. Note further that in an exemplary embodiment, device 1699 can include a microphone or some other form of sound capture device. It is noted that device 6099 as described in this embodiment is a device for sensing phenomenon associated with an epileptic seizure. It is noted that in an alternative embodiment, device 1699 can stand in for the blood glucose sensors detailed above, or other types of sensors. It is also noted that in an exemplary embodiment, the implanted component such as device 1299 detailed above, it could also be used, and thus the disclosure of device 1699 also corresponds to an alternate disclosure of the device 1299 alone or with an external component such as the BTE device detailed above, as well as an alternate disclosure that discloses both device 1699 and device 1299 with or without the BTE device.

It is briefly noted that while the embodiments disclosed in the figures show two-way communication, in some embodiments, there is only one-way communication. Accordingly, in at least some exemplary embodiments, any link disclosed herein showing two-way communication corresponds to an alternate disclosure where there is only one-way communication providing that the art enables such. Also seen in FIG. 16 is a device 1616 which is in the form of an inhaler, which in this exemplary embodiment, is configured for wireless communication with the devices a shown, and can be programmable. More on this in a moment, but as seen in FIG. 16, there can be direct communication from the master (device 1699) to the slave (the inhaler 1616) drug delivery device. Still further as can be seen, there can be indirect communication between the device 1699 and the inhaler 1616 via the cloud. Still further as can be seen, there can be an indirect connection, where the connection between device 6099 and inhaler 1616 is bridged by the smart phone 2401 or a non-smart phone, such as a traditional phone.

In an exemplary embodiment, some types of information that can be exchanged over one or more of the links shown in FIG. 16 can be a patient identifier, which can be a unique patient (user) identifier that can be exchanged with the drug delivery system (inhaler 1616) to ‘unlock’ the drug delivery device for a particular user (as opposed to another user who might utilize the inhaler 1616, where a replaceable and/or disposable mouthpiece can be utilized, in the case of a shared inhaler). Thus, in an exemplary embodiment, there is a first patient that utilizes a first sensor 1699, and the second patient that utilizes a second sensor 1699, and depending on the user identifier that can be stored in the pertinent sensors, the inhaler 1616 will provide a particular therapeutic substance in a particular amount for one patient relative to the other patient. Note that more than two patients can utilize the system. And it is also noted that while the term patient identifier is utilized herein, that phrase can be a proxy for the device 1699 identifier, where it is the device identity that can control (although in some embodiments, the sensor 1699 can be configured to sense certain features that will enable the sensor to identify a particular person or otherwise differentiate one person from another person).

Also, therapeutic substance type and/or dosage can be transmitted. In an exemplary embodiment, the patient identifier references the relevant drug information for the individual accessing the system. Also, temporal data can be transferred, such as the utilitarian or otherwise desired timing. This timing can be exchanged in an effort to increase the utilitarian aspects of the delivery of the therapeutic substances, at least in the case where the master believes it is not the optimal time. In this regard, the therapeutic substance delivery system can be controlled to prohibit the delivery of the therapeutic substance.

Compliance data can be transferred. For example, the inhaler 1616 can exchange with the master device data indicative of whether or not the patient has taken the drug. In the case of this inhaler, the dosage that was administered can be transferred and/or the type a therapeutic substance that was administered can be transferred. In an exemplary embodiment, the inhaler can be configured to log this data such as for example by determining the number of times that a button was pressed (or whether the button was pressed in the first instance for that matter).

In view of the above, it can be seen that in an exemplary embodiment, there is an exemplary system as detailed above, where the system includes a second medical device configured to provide a therapeutic substance to the person; the first medical device is the sensor. With respect to the system where the first medical device includes data indicative of past treatment, the data indicative of past treatment includes data from the second medical device pertaining to compliance of therapeutic substance usage of the person by the second medical device. This can be communicated via any of the links detailed herein. Based on the past compliance, the first medical device can determine whether or not it is utilitarian for the therapeutic substance delivery device to deliver therapeutic substance and/or in what amounts and/or what type of therapeutic substance. If compliance was poor with one type a therapeutic substance, another type of therapeutic substance might be utilitarian or otherwise might evoke better compliance. Still further, the data indicative of past treatment that is stored in the first medical device can include data from the second medical device pertaining to previous amounts of drug delivery provided to the person by the second medical device. Further, the first medical device can be configured to prohibit the delivery of at least one therapeutic substance from the second medical device for at least a limited time based on the data indicative of past treatment during a time that would otherwise include delivery.

Also, error data can be transferred. By way of example only and not by way of limitation, in a scenario where an error occurs in the delivery of the drug, information indicative of such can be exchanged back to the master device.

Biosensors and/or model outputs can be transferred. The cloud communication can be used, or the information can be transferred directly from the master device to the inhaler. Moreover, new information relating to changes to the drug dose and/or timing of administration can be communicated based on biosensor inputs and/or predictive models that are running for each patient or patient groups. These revised doses and/or timing calculations can be done in an effort to obtain utilitarian treatment for a given individual.

With respect to inhaler 1616, the inhaler can be an aerosoliser. The inhaler can be configured to provide different dosages and/or different therapeutic substances. In an exemplary embodiment, the inhaler includes different types of therapeutic substances in different amounts, located in reservoirs or containers therein. In an exemplary embodiment, the inhaler includes control unit such as a processor or logic circuitry, that is configured to receive input, such as input from the various components shown in FIG. 16, and determine what type of therapeutic substance and/or how much of that therapeutic substance should be delivered when the inhaler is to be utilized. In an exemplary embodiment, the inhaler includes actuators and electromechanical devices that can be controlled by the logic circuitry to open or otherwise present a specific therapeutic substance in a specific amount for inhalation.

Again, it is noted that in at least some exemplary embodiments, the teachings detailed herein can be utilized to actually stop the overconsumption and/or improper timing of therapeutic substance delivery/use. In this regard, as noted above, in some embodiments, the inhaler will not dispense therapeutic substance unless the sensor 1699 affirmatively permits the inhaler to operate. Conversely, in some other embodiments, the inhaler itself will only operate during certain time periods and/or dispense certain amounts based on instructions that it received previously, such as minutes and/or hours and/or days, prior to the action of dispensing, where the inhaler implements the control based on the aforementioned instructions. In this regard, it is a smart slaved device.

Accordingly, as can be seen, in an exemplary embodiment, the dispensing device can be programmable. In an exemplary embodiment, the sensor 1669 programs the dispensing device.

Fast acting drug delivery systems that target specific diseases which have uncertain onsets and often significant clinical impact (such as epilepsy) can be used as the therapeutic substance delivery device. These devices can be used in emergency clinics and/or at home with several users. In the case of ingested therapeutics, such devices can have the ability to swap the mouthpiece and adjust the drug dosage (an in some embodiments, apply different type of drug). This is a feature that has utilitarian value with respect to embodiments where, for example, an inhaler or other therapeutic substance delivery system is shared by others (such as the pill dispenser detailed herein). In the case of injected therapeutics, such devices can have a quick attachment termination so that a given smart syringe can be utilized over multiple patients. In this regard, in an exemplary alternate embodiment, instead of the inhaler 1616 in FIG. 16, a smart syringe can replace the inhaler, with the overall concept of the embodiment of FIG. 16 remaining.

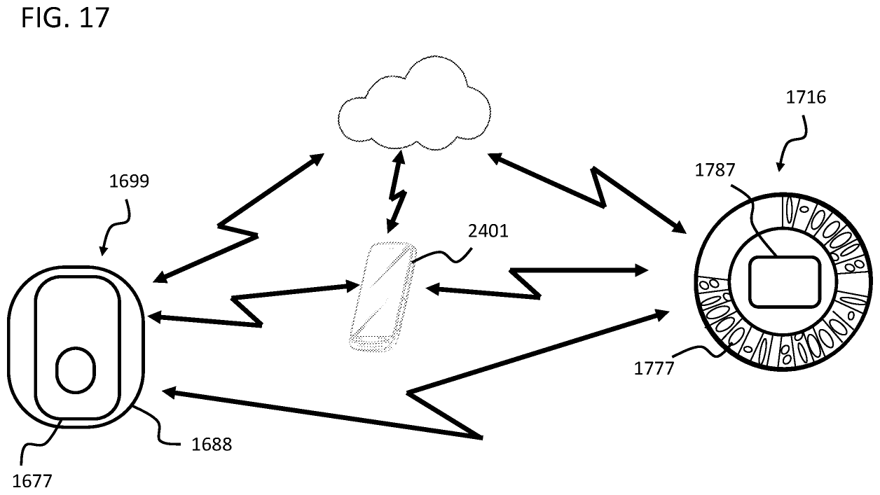

While the embodiment of FIG. 16 was directed towards a system for the treatment of epilepsy, in an alternate embodiment, the embodiment of FIG. 16 can correspond to a system for the treatment for diabetes. Sensor 1699 can instead be a blood glucose monitor, which can be a continuous monitor, such as the patch embodiment described above. Also, instead of inhaler 1616, the transdermal patch 1110 can be utilized as the therapeutic substance delivery system. That said, in an exemplary embodiment, instead of an injection or transdermal system for the therapeutic substance delivery, a pill based therapeutic substance delivery system can be utilized. In this regard, FIG. 17 presents an exemplary embodiment that utilizes a smart pill dispenser 1716. Here, there are a plurality of different pills 1777 located in different chambers. The smart pill dispenser 1716 includes a control unit 1787, which can be a processor based or HF-based or otherwise can correspond to logic circuitry, configured to receive input from a user and also receive input from the various other components shown in FIG. 17, and utilize such input to dispense one or more therapeutic substances from a plurality of therapeutic substances stored in the smart pill dispenser. Also, different quantities can be dispensed. This can have utilitarian value, by way of example only and not by way of limitation, in a household where two or more people suffer from diabetes, which people can be treated utilizing the pill based therapeutic substances (the dispenser can be a shared dispenser). Accordingly, in an exemplary embodiment, a first person wearing a first sensor device 1699, can hold the pill dispenser 1716, and the first sensor device 1699 can communicate the identifying information to the pill dispenser 1716, along with one or more of the other data noted above, and the pill dispenser 1716 can receive this data, and prepare certain pills for dispensation. The person then activates the pill dispenser to dispense pills, and the pill(s) that were prepared for dispensation are dispensed. The pill dispenser then records this action and/or provides data back to the sensor 1699 to indicate that the first person has taking these pills (based upon the latent variable that the pill dispenser has dispensed pills—in an alternate embodiment, the pill dispenser can be a device that requires affirmative input from the person utilizing the pill dispenser that he or she has actually taken the pills—the pill dispenser can be configured to receive input from the first person). This process can be repeated for the second person, where data indicative of the identification of the second person is transmitted from a second sensor 1699 for that second person, to the same pill dispenser that was utilized by the first person. This can be the case for three or four or five or six or more people.

In view of the above, it can be seen that the teachings detailed herein can correspond to various embodiments of delivery modes such as, for example, smart devices, which can include smart inhalers, smart pill dispensers, smart transdermal patches/delivery devices. The delivery modes can also include smart syringes. Of course, delivery can be executed utilizing traditional syringes, oral drugs/pills, etc. In some embodiments, the teachings detailed herein are directed towards obtaining or otherwise devising a prescription for a given chemical substance. Embodiments can be such that the medical device at issue is configured to communicate with the delivery mode, which can be any one or more of the offer mentioned modes, and is configured to exchange information pertaining to the user of the medical device, such as, for example, current drug compliance and/or data relating to efficacy. The “delivery mode” can then decide on the appropriate does or prescription. In some embodiments, a healthcare professional who is licensed can then in person or remotely review and sign off on the developed treatment modality.

The concepts of FIGS. 16 and 17 can be applicable to other types of shared medical devices, such as by way of example only and not by way of limitation, a transdermal patch application device. In an exemplary embodiment, a handheld application device can be utilized to apply the various patches detailed herein and variations thereof, and other types of patches for that matter. With respect to the overall system, the inhaler 1616 would be replaced with this handheld application device.

In view of the above, can be seen that embodiments can address the complex issue of having multiple users associated with the same delivery device. The teachings detailed herein can be implemented so as to improve the likelihood or otherwise ensure the appropriate drug or dosage is prescribed for the individual interacting with the delivery system. Here, it can be seen that the drug delivery systems go beyond the mere prescriptive nature of devices that do not have the communication regimes detailed herein. The embodiments disclosed herein can affirmatively take into account an individual's current disease/condition. It is noted that the embodiments of FIGS. 16 and 17 can be combined with the embodiments above with respect to FIG. 12, for example, and other embodiments detailed herein for that matter.

In view of the above, it can be seen that in an exemplary embodiment, the closed-loop system can further include a therapeutic substance delivery device to deliver future therapeutic substance(s), wherein the therapeutic substance delivery device includes a plurality of different therapeutic substances for different users of the therapeutic substance delivery device. This can be two or three or four or five or more users. In an exemplary embodiment, the therapeutic substance delivery device is configured to automatically determine which of the different therapeutic substances are to be delivered depending on the user of the therapeutic substance delivery device. For one user, certain therapeutic substance will be delivered, and for another user, another therapeutic substance will be delivered. Moreover, as can be seen, the therapeutic substance delivery device is controlled by the implanted and/or body worn component to determine which of the different therapeutic substances are to be dispensed depending on the user of the therapeutic substance delivery device.

Still further, in embodiments where the system further includes a therapeutic substance delivery device to deliver the future therapeutic substance(s), the therapeutic substance delivery device is configured to dispense therapeutic substance(s) based on a control regimen stored in the delivery device (amount and/or type), the implanted and/or body worn component has previously established the control regimen stored in the delivery device, and the implanted and/or body worn component is configured to establish a new control regimen to be stored in the delivery device. This new control regimen can supersede the prior control regimen for the existing control regimen.

Returning back to embodiments that utilize hearing prostheses devices, in some exemplary embodiments, inclusive of cochlear implants, bone anchored implants, middle ear implants and/or vestibular devices, the following can be applicable:

| Data Stored | | | |
|---|---|---|---|
| and/or | | | |
| Exchanged | Origin | Some exemplary scenarios | Exemplary Application |
| evoked potentials | sensor | The implant has been | The implant can stream the |
| and electrode | | successfully implanted and | evoked potentials and/or |
| voltages from the | | the surgeon or other | impedances to determine if |
| cochlea (during | | healthcare professional seeks | the drug is being |
| surgery) | | to apply a particular drug | administered in the correct |
| | | therapy directly to the | location and/or whether the |
| | | cochlea. | drug is having the desired |
| | | | effect |
| one or more | memory + | The implant or other part of | To assess if drug treatment is |
| historical | sensor | the system of which the | currently effective at slowing |
| audiograms, | | implant is a part stores the | or improving hearing loss. To |
| evoked potentials | | historical patient information | advise the physician to |
| and/or | | in, for example, an effort to | adjust or modify the dose. |
| impedances | | determine if changes to the | |
| | | hearing of the individual has | |
| | | occurred. | |
| patient reported | subjective | Patient reported information | This information can be |
| information | | relating to their own | utilized to exchange with the |
| | | assessment of either their | treating physician (along |
| | | hearing quality, variability, | with the sensors) to “close |
| | | etc. and/or their well being | the loop” on the drug |
| | | e.g., vertigo, balance issues | treatment and whether the |
| | | etc. | prescription needs to |
| | | | change. |

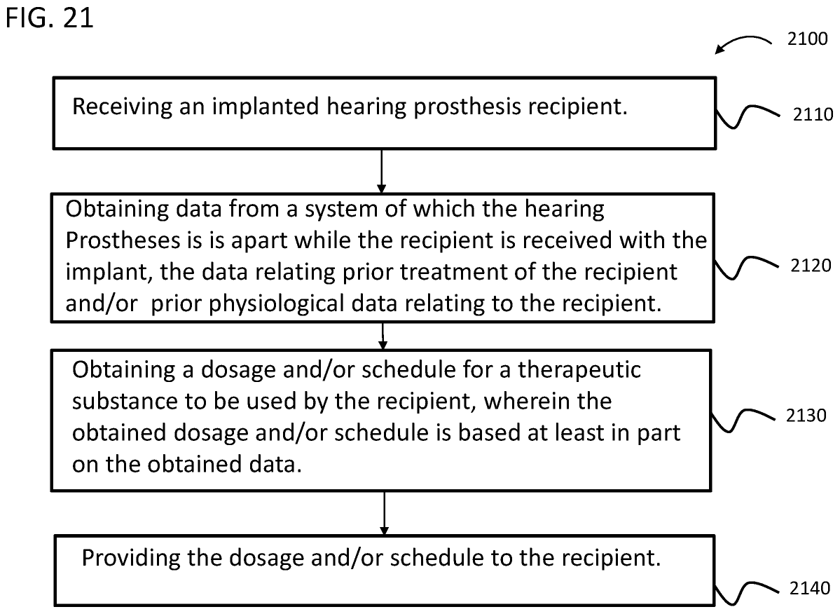

FIG. 21 presents a flowchart for an exemplary method, method 2100. This method is directed towards people who have been implanted hearing prostheses, as distinct from, for example, a conventional hearing aid, which is not implanted. It is noted that a recipient with an implanted hearing prosthesis can also include a conventional hearing prosthesis such as can be the case with respect to a bimodal hearing prosthesis.

Method 2100 includes method action 2110, which includes receiving an implanted hearing prostheses recipient. In an exemplary embodiment of method action 2110, the action of receiving results from the recipient visiting a hearing prostheses clinic or otherwise a hearing prostheses specialist, or a location that sells hearing prostheses along with other arrangements (conventional hearing aids are ubiquitous in so-called big-box stores). In an exemplary embodiment, the action of receiving implanted hearing prostheses recipient is executed at a location that has revenue primarily relating to conventional hearing aids (revenue from sales and/or from support for such devices, etc.). In an exemplary embodiment, on a per quantity basis, at least 50, 60, 70, 80, or 90% of a per unit sales base corresponds to the sale of conventional hearing aids with respect to the overall hearing prostheses sold at this location. In an exemplary embodiment, the affirmation qualifiers existed for 1, 2, 3, 4, 5, 6, 7, 8, 9, 10, 11, 12, 13, 14, or 15 fiscal years, or any range of time periods therein in one year increments within a time period of at least 1, 2, 3, 4, 5, 6, 7, 8, 9, 10, 11, 12, 13, 14, or 15 calendar years or any range of time periods therein in one year increments prior to the action of receiving an implanted hearing prestigious recipient. Thus, for example, in the year 2021, the location could be a location where, in the years 2016, 2017 and 2018, the majority of the sales of hearing prostheses on a per unit basis relative to all hearing prostheses sold was at least 60%.

Method 2100 further includes method action 2120, which includes the action of obtaining data from a system of which the hearing prosthesis is apart while the recipient is received with the implant, the data relating prior treatment of the recipient and/or prior physiological data relating to the recipient. In an exemplary embodiment, the data can be obtained from, for example, memory in a behind-the-ear device or memory in an off the ear device that is part of the hearing prostheses system. In an exemplary embodiment, the data can be obtained from an implanted memory implanted in the recipient. In an exemplary embodiment, the data can be obtained from device 2401 for example, if that device is part of a system of which the hearing prostheses is apart. In this exemplary embodiment, the system is confined to that which is implanted in the recipient, worn on the recipient or otherwise in the immediate possession of the recipient without the need to access a remote device or remote memory remotely located from the location. In some embodiments of method 2100, prior to the action of receiving of method action 2110, the recipient has received a therapeutic substance, and a system of which the hearing prosthesis is a part has recorded therein data relating to the received therapeutic substance. In some embodiments, the system automatically recorded therein data relating to the received therapeutic substance. This automated recording can be in accordance with the teachings detailed above. Conversely, in some embodiments, the data was manually imported into the system.

In an exemplary embodiment, the data can correspond to any of the data detailed herein and other data that can have utilitarian value. In an exemplary embodiment, the hearing prostheses is configured to obtain the data relating to prior physiological data. This can be done, for example, utilizing the electrodes of a cochlear implant electrode array for sentencing purposes, as detailed above with respect to the cochlear implants invented by Paul Carter and John Heasman detailed above. In an exemplary embodiment, the data can be obtained by other devices, and loaded or otherwise logged by the hearing prostheses system.

Returning back to FIG. 21, method 2100 further includes method action 2130, which includes the action of obtaining a dosage and/or schedule for a therapeutic substance to be used by the recipient, wherein the obtained dosage and/or schedule is based at least in part on the obtained data. This aspect has been detailed above with respect to other types of medical devices, and thus the above concepts are to be extrapolated to the hearing prostheses specific embodiment detailed herein in the interest of textual economy. In an exemplary embodiment, the action of obtaining the dosage and/or schedule is executed in conjunction with a licensed healthcare professional who is legally allowed to spur scribe certain medications. This can be executed remotely, where the data is provided to the healthcare professional, such as via the cloud or via a phone line, etc., who then analyzes the data and then identifies the dosage and/or schedule for the therapeutic substance to be used. This data is then communicated back to the location where the recipient is located (or was located, it can be communicated after the recipient has left the location). Conversely, in an exemplary embodiment, the various computing techniques are logic techniques detailed herein could automatically or semiautomatically identify the dosage and/or schedule for the recipient, and provide such to the location. In an exemplary embodiment, the logic that is utilized to identify the dosage and/or schedule can be located at that location, while in other embodiments, the logic could be located at a remote location. In this regard, in an exemplary embodiment, this parallels the second subsystem detailed above.

Method 2100 further includes method action 2140, which includes providing the dosage and/or schedule to the recipient. It is noted that this can be executed by providing the dosage and/or schedule directly to the recipient, or by providing the dosage and/or schedule to a medical device of the recipient, which may or may not be the hearing prostheses. In this regard, some embodiments can be executed where the recipient does not have direct knowledge of the dosage and/or schedule. This can still be utilitarian because the devices detailed herein can operate autonomously without the recipient knowing exactly what is going on or even knowing anything about what is going on. Still, in alternative embodiments, as noted above, affirmative action is required on the part of the recipient, so in some instances, method action 2140 is executed with the knowledge of the recipient.

In an exemplary embodiment, the recipient that is the subject of method 2100 previously was a conventional hearing aid recipient only (in that the person did not have an implanted hearing prosthesis—here she may have had another type of implant). In an exemplary embodiment, method 2100 further includes, prior to the action of receiving the implanted hearing prosthesis recipient, receiving the recipient while the recipient is a recipient only of the conventional hearing aid and providing a second dosage and/or schedule for a therapeutic substance to the recipient. That said, in an exemplary embodiment, prior to the action of receiving, the method could include receiving the recipient while the recipient is a recipient only of the conventional hearing aid, and providing an adjustment or otherwise adjusting the setting of the conventional hearing aid. Indeed, in an exemplary embodiment, method 2100 further includes, prior to the action of receiving the implanted hearing prostheses recipient, receiving the recipient prior to the recipient being a recipient of only the conventional hearing aid, and providing the recipient with the conventional hearing aid. Note that in an exemplary embodiment, it may be the case of the recipient previously had a conventional hearing aid, but this is not the conventional hearing aid at issue in this method. That said, in some embodiments, the conventional hearing aid is the first hearing aid the recipient has ever had. In an exemplary embodiment, this can occur 1, 2, 3, 4, 5, 6, 7, 8, 9, or 10 years or more before the method actions of method 2100.

In an exemplary embodiment, the obtained data is data based on historical usage of the hearing prosthesis and/or hearing capabilities of the recipient stored in a system of which the hearing prosthesis is a part and/or data relating to a therapeutic substance taken by the recipient. In an exemplary embodiment, the historical usage goes back 1, 2, 3, 4, 5, 6, 7, 8, 9, 10, 11, 12, 13, 14, 15, 20, 25, 30, 35, 40, 45, 50, 60, 70, 80, or 90 months or more, or any value or range of therebetween in one month increments. In an exemplary embodiment, the obtained data relates to prior physiological data relating to the recipient and the prior physiological data is based on data obtained by a medical device worn and/or implanted in the recipient. In this exemplary embodiment, the medical device could be the hearing prosthesis, and in an alternate embodiment, it can be another device. As noted above, sensing devices completely separate from the underlying stimulating device are envisioned in at least some exemplary embodiments.

In some instances, the action of providing the dosage and/or schedule to the recipient results in a completely new therapeutic substance to be provided to the recipient. By way of example only and not by way of limitation, the recipient may not be on a medication or a therapeutic substance, or is on a different therapeutic substance, and the results of method 2100 is that the recipient is taking a new medication for example. In an exemplary embodiment, the qualifier relating to the completely new therapeutic substance is limited to that which is the case with respect to the time period preceding the action of receiving that extends back at least 1, 2, 3, 4, 5, 6, 7, 8, 9, 10, 11, 12, 13, 14, 15, 20, 25, 30, 35, 40, 45, 50, 60, 70, 80, or 90 months or more, or any value or range of values therebetween in one month increments. By way of example only and not by way of limitation, in an exemplary embodiment, if an anti-inflammatory medication was originally given to the recipient at the time of implant, and three or four years have passed since the time of implantation and the action of receiving, and the recipient has not utilize this anti-inflammatory medication in those intervening years, the therapeutic substance can be considered new if qualified by the aforementioned time periods.

It is noted that some exemplary embodiments of method 2100 can be expanded to the actual action of providing the therapeutic substance to the recipient. In some embodiments, depending on the regulatory environment, this can be executed at the location where the recipient is received. In some embodiments, this is decidedly not executed at that location. Instead, a traditional pharmaceutical dispensary is needed to execute this method action. That said, consistent with the teachings detailed herein, it can be that the recipient is already in possession of the therapeutic substance, such as by way of example only and not by way of limitation, because the recipient is wearing and/or has implanted therein were otherwise attached thereto the therapeutic substance delivery device detailed above or the recipient has the aforementioned inhaler or the therapeutic substance delivery device in the form of the pill dispensary, etc. It could be that the therapeutic substance is not readily accessible to the recipient owing to the various control regimes detailed herein. In this regard, it could be that the system of which the hearing prosthesis is a part must communicate to the therapeutic substance delivery device that it is now permissible to provide the therapeutic substance to the recipient. This is consistent with the concept that the implantable device can be a master and the therapeutic substance delivery device could be a slave.

In an exemplary embodiment, the data stored, exchanged and/or the clinical application/use for the data in the system that is included in the sensory medical device can be utilized in a hearing therapy environment or otherwise used in conjunction with hearing prostheses. The after mentioned data can relate to vestibular functionality and/or the treatment thereof or otherwise monitoring associated there with. As will be detailed below, the data can relate to cardiac related treatment or otherwise monitoring, where in an exemplary embodiment, the stimulating device is a pacemaker, and the sensory device is an EKG sensory device. Consistent with the above, the data can also relate to the treatment of epilepsy, whether such entails treating a person who is experiencing a seizure, or otherwise minimizing the likelihood that a seizure will exist. With respect to some general background of the sensing devices detailed herein, it is again noted that in at least some exemplary embodiments, the medical device can be configured to execute EEG monitoring. In an exemplary embodiment, the devices and systems herein are configured to implement data collection activity in general, and EEG signal collection/recording/reading in particular. As will be detailed below, the data collection activity can instead be EKG signal collection/recording/reading. The data collection activity can also be single action potentials, multi-unit cluster recordings, compound action potentials, or other neural responses. Other types of data collection activities can be utilized. Any data collection activity that can have utilitarian value with respect to analyzing a feature associated with the body of the recipient can be utilized in at least some exemplary embodiments. Such readings can be utilized in the monitoring of one or more physiological parameters of the user of the medical device of the closed-loop monitoring system detailed above.

Concomitant with the embodiments detailed above associated with the EEG/EKG monitoring, in an exemplary embodiment, the data collection activity is data collection utilizing an implanted electrode that is part of the medical device. It is noted that in at least some exemplary embodiments, electrodes of the cochlear electrode array can be utilized. These electrodes can be the electrodes that are implanted in the cochlea or extra cochlear electrodes—any electrodes that are utilized to evoke a hearing percept can be used in some embodiments. As just noted, the electrode(s) can also be the extracochlear electrode that is used for the return in monopolar stimulation (e.g., the so-called ball electrode, or the plate electrode which is located on the receiver stimulator of the cochlear implant, etc.). These also can be additional electrodes that are added to the cochlear implant electrode array, such as electrodes arrayed as seen in FIG. 5, which are integrated with or otherwise in communication with the cochlear implant.

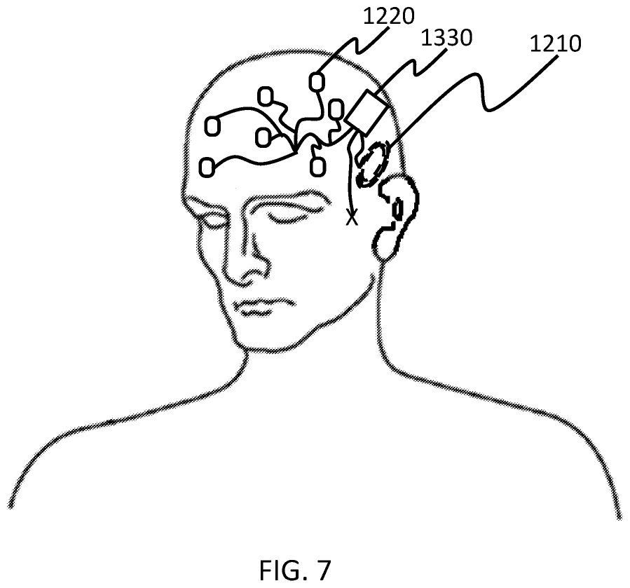

Along these lines, FIG. 7 presents an exemplary embodiment of a modified version of the embodiment of FIG. 6 detailed above. In this embodiment, which is presented in functional conceptual terms (e.g., coil 1201 and housing 1330 would be part of an integrated assembly), a cochlear implant is represented with the cochlear implant electrode array represented by the “X.” This is an example of how a cochlear implant electrode array can be integrated with an EEG read electrode apparatus. In this embodiment, the housing 1330 can include the implanted circuitry and components of a cochlear implant electrode array, which can be modified to have the functionality for the purposes of EEG reading or can instead also include in that housing separate circuitry for the purposes of EEG reading.

It is also noted that FIG. 7 conceptually represents different types of hearing prostheses than a cochlear implant. The middle ear actuator can be represented by the “X,” or the implanted actuator of a bone conduction device can be represented by the “X,” and the housing 1330 can include the circuitry that is utilized to control the implanted actuator, while also including house there in the circuitry that can enable the EEG reading.

It is also noted that in at least some exemplary embodiments, housing 1330 can include a speech processor or the like, such as that which would be implanted in the case of a totally implantable hearing prosthesis.

It is noted that herein, the phrase recipient is sometimes utilized. Any disclosure herein that refers to the recipient corresponds to an equal disclosure of a person whether or not that person is a recipient of a prostheses, unless otherwise noted, providing that the art enables such, and vice versa.

In an exemplary embodiment, one or more of the devices and/or systems and/or subsystems, etc., disclosed herein, and variations thereof, include a processor, which processor of can be a standard microprocessor supported by software or firmware or the like that is programmed to execute one or more of the actions and functionalities herein. The processor can include input and/or output connections. By way of example only and not by way of limitation, in an exemplary embodiment, the microprocessor can have access to lookup tables or the like having data and/or can compare features of the input signal and compare those features to features in the lookup table, and, via related data in the lookup table associated with those features, make a determination about the input signal, and thus make a determination, etc. Numeric analysis algorithms can be programmed in the processors, etc., to implement the teachings herein.

It is noted that the teachings detailed herein can be implemented in any processor-based device that can enable the teachings herein. In an exemplary embodiment, a sensory prosthesis, such as a hearing prosthesis or a light prosthesis, can be modified by adjusting the circuitry or otherwise providing programming to a given processor so as to enable the teachings detailed herein. Further, an Internet of things-based approach can be utilized. Also, various components and systems and subsystems can be network so that some actions and/or functionalities detailed herein are performed by components that are remote and/or geographically distant from other components. Accordingly, the teachings detailed herein can be implemented utilizing the Internet or landline-based devices or wireless communication system such as cellular phone communication systems, etc. Any of the prostheses and/or medical devices detailed herein can correspond to body worn devices or body carried devices. Again, these body worn or body carried devices can have processors that are programmed to receive input and/or to provide output to implement the teachings detailed herein. In some embodiments, programs personal computers and/or laptop computers and/or personal handheld devices, such as smart phones or smart watches etc. can be utilized to execute at least some of the functionalities and method actions detailed herein.

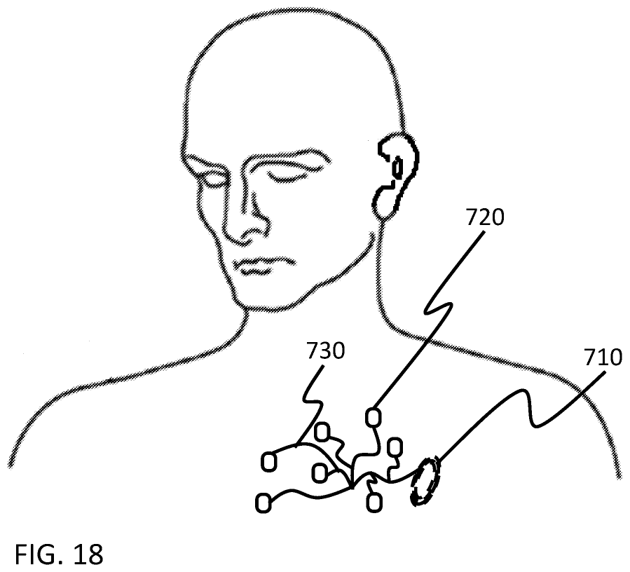

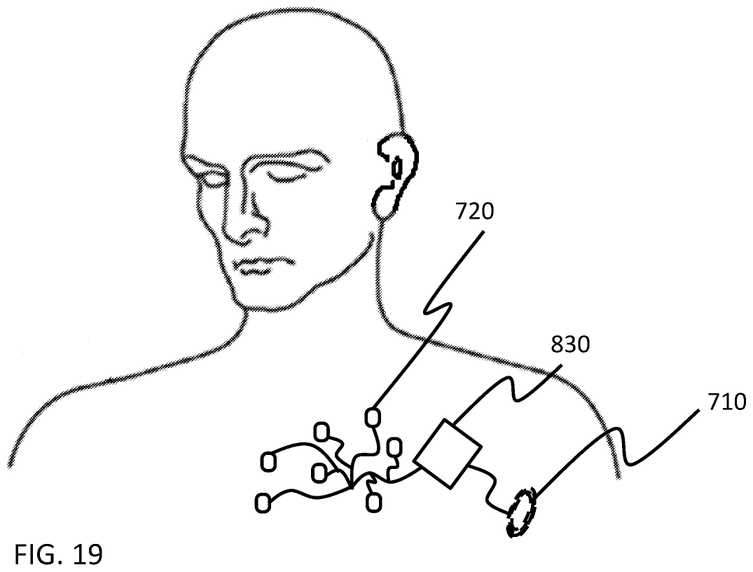

Many of the embodiments detailed above have focused on a device that is implanted in the head or otherwise includes an inductance coil that is located in the head. Some other embodiments can be embodiments that include an implanted component that is implanted elsewhere other than the head. By way of example only and not by way of limitation, in an exemplary embodiment, there can be a heart monitor and/or a heart stimulator (pacemaker), such as by way of example only and not by of limitation, the arrangement seen in FIG. 18. As seen, a heart monitor comprises a plurality of sensor/read electrodes 720, connected to an inductance coil 710 via leads 730. In this embodiment, the implanted device has no recording/storage capabilities, and requires an external device to receive a signal from the implanted inductance coil 710 so as to retrieve in real time the signal therefrom. Not shown is an implantable component that converts the electricity sensed by the sensor/read electrodes into a signal that is transmitted by the inductance coil 710. In an exemplary embodiment, the sensor arrangement seen in FIG. 7 is an implanted EKG sensor arrangement. FIG. 19 depicts another arrangement of an implantable sensor arrangement that again includes the sensor/read electrodes 720 and the leads 730. Here, in this embodiment, there is a housing 830 which includes circuitry that is configured to receive the signals from the leads from the electrodes 720 and record the data therefrom or otherwise store the data, and permits the data to be periodically read from an external device when the external device comes into signal communication with the implanted inductance coil 710. Alternatively, and/or in addition to this, the circuitry is configured to periodically energize the inductance coil 710 so as to provide the data to the coil 710 so that it creates an inductance signal which in turn communicates with an external component that reads the signal and thus reads the data associated with the electrodes. Thus, in at least some exemplary embodiments, the implantable apparatus is configured to stream the data. Still further, in some embodiments, the data is not streamed, but instead provided in bursts.

Embodiments can be used with/can include any device that stimulates the heart to return the pulse rate to normal rate, implantable cardioverter defibrillators (ICDs), devices that stimulate the heart to stop action potentials responsible for abnormal rhythms, cardiac resynchronization therapy (CRT), biventricular pacemakers that stimulates both left and right ventricles to make contract at the same time, etc. In some scenarios of the prior art, some patients with either PM, CRT or ICDs will experience inappropriate stimulations/shocks from the device due to medications that are cardio active in nature. These cardioactive drugs can decrease or increase the defibrillation threshold and/or decrease or increase the excitation of the heart. In some scenarios, the drugs that cause the effects described above can be classified according to the Vaughan-Williams classification in class I-V drugs. It is also possible to administer drugs to protect against inappropriate shocks, such as calcium channel blockers. These can be administered to the patient to counteract other drugs or to stabilise conditions to ensure correct pacemaker operation. Accordingly, in some exemplary embodiments, the teachings detailed herein are implemented so as to control or otherwise prevent or encourage as applicable the aforementioned effects.

Below is an exemplary chart relating to some exemplary embodiments that can be used in conjunction with the teachings herein:

| Data Stored | | | |
|---|---|---|---|
| and/or | | | |
| Exchanged | Origin | Some exemplary scenarios | Exemplary Application |
| Brain activity | sensors | monitoring of | monitoring of the |
| (EEG), heart | and/or | baseline brain | incorrect stimulation / |
| (ECG), | subjective | activity and events | shocks to the heart |
| respiration and | | (such as hypoxic | monitoring of the drug |
| blood oxygen | | events or | efficacy of the drug |
| saturation | | desaturations due to | treatment (using the |
| (SpO2) | | cardiac malfunction) | subjective and objective |
| | | monitor the vital | information). This data |
| | | signs of the patient | is then fed back to the |
| | | and detect any | cloud or the drug |
| | | abnormalities around | delivery device (or |
| | | respiration, cardiac | treating physician) |
| | | function etc. | via the vital sign |
| | | | monitoring provide |
| | | | objective assessment of |
| | | | drug side effects (i.e., |
| | | | shocks) and adjustments |
| | | | to the drug treatment |
| | | | that may yield less |
| | | | incorrect shocks to the |
| | | | heart |
| historical of | memory + | The implant stores the | To assess if drug treatment is |
| drug use and | sensor | historical patient | currently effective at |
| efficacy | | information in an effort to | minimising the incorrect shocks. |
| | | exchange this to optimise | To advise the physician to |
| | | the treatment of the | adjust or modify the dose |
| | | individual | |
| patient | subjective | Patient reported | This information can be utilised |
| reported | | information relating to their | to exchange with the treating |
| information | | own assessment of either | physician (along with the |
| | | their well-being, incorrect | sensors) to ‘close the loop’ on |
| | | stimulation or shocks and | the drug treatment and |
| | | cognitive function. | whether the prescription needs |
| | | | to change. See above. |

Some embodiments of the teachings detailed herein are applicable to sleep apnea devices and methods of treating such. In this regard, some exemplary embodiments are related to implantable devices for the treatment of obstructive sleep apnea. Some embodiments include identifying or otherwise working with drugs that can be taken by individuals for other conditions that impact either positively or negatively OSA. Some drugs that treat OSA can be drugs that act on specific root causes of OSA, such as muscle tone, breathing, low arousal threshold, etc. in some instances, there can be difficulties with these drugs with regard to finding the right treatment does in that one or more drugs may improve an aspect of OSA but either worsen or fail to address other aspects. By way of example only and not by way of limitation, a drug therapy may reduce the blockages of the airways but not address the regular wake periods of the person experiencing OSA. Moreover, some side effects of the drugs may counterbalance other aspects of sleep such as a REM (deep sleep) and the drowsiness the next day. Long-term monitoring can in some instances be required of the patient to quantitatively assess the sleep patterns, respiration, body movement and subjective impact during awake hours.

In an exemplary embodiment, the treatment of OSA is linked with the treatment of epilepsy. In an exemplary embodiment, there is a system that combines both monitoring and therapy that is directed towards patients with epilepsy to treat OSA. In this regard, the population that only has OSA is different and distinct from the population that has epilepsy and also OSA. Thus, exemplary methods include one or more the method actions detailed herein utilized with respect to a human being that suffers from epilepsy and also OSA. With respect to the method actions detailed herein regarding monitoring, the monitoring relating to OSA is refined to address the fact that the patient has epilepsy. With respect to method actions detailed herein regarding treatment of OSA, the treatment is refined to address the fact that the patient has epilepsy.

In an exemplary embodiment, efficacy of the treatments is measured. In some exemplary embodiments, efficacy is not just measured as the reduction/treatment of OSA. Efficacy is also measured by the minimization or otherwise the reduction of the deleterious impact on sleep quality and/or sleep disorders.

Below is an exemplary chart relating to some exemplary embodiments that can be used in conjunction with the teachings herein. Such embodiments can, in some instances, alleviate one or more or all of the aforementioned difficulties.

| Data Stored | | | |
|---|---|---|---|
| and/or | | | |
| Exchanged | Origin | Some exemplary scenarios | Exemplary Application |
| Brain activity | sensors | monitoring of | monitoring of the |
| (EEG), heart | and/or | baseline brain | incorrect activation of |
| (ECG), | subjective | activity and events | the sleep apnea |
| respiration and | | that are related to | stimulation |
| blood oxygen | | sleep (such as sleep | monitoring of the drug |
| saturation | | spindles and sleep | efficacy of the drug |
| (SpO2) | | stage cycling) | treatment (using the |
| | | monitor the vital | subjective and objective |
| | | signs of the patient | information). This data is |
| | | and detect any | then fed back to the |
| | | abnormalities | cloud or the drug |
| | | around respiration, | delivery device (or |
| | | cardiac function etc. | treating physician) |
| | | | via the vital sign |
| | | | monitoring provide |
| | | | objective assessment of |
| | | | drug side effects and |
| | | | adjustments to the drug |
| | | | treatment that may yield |
| | | | improved delivery of the |
| | | | therapy |
| historical of | memory + | The implant stores the | To assess if drug treatment is |
| drug use and | sensor | historical patient | currently effective at minimising |
| efficacy | | information in an effort to | sleep abnormal events or good |
| | | exchange this to optimise | sleep. To advise the physician to |
| | | the treatment of the | adjust or modify the dose |
| | | individual | |
| patient | subjective | Patient reported | This information can be utilised |
| reported | | information relating to their | to exchange with the treating |
| information | | own assessment of either | physician (along with the |
| | | their well-being, sleep | sensors) to ‘close the loop’ on |
| | | quality and cognitive | the drug treatment and |
| | | function. | whether the prescription needs |
| | | | to change. See above. |

Any arrangement that can enable the data associated with the read electrodes to be provided from inside the recipient to outside the recipient can be utilized in at least some exemplary embodiments. In this regard, traditional implanted EKG sensor arrangements can be obtained and modified so as to implement the teachings detailed herein and/or variations thereof.

In view of the above, it can be seen that the aforementioned measurements can also correspond to EKG measurements or the like.

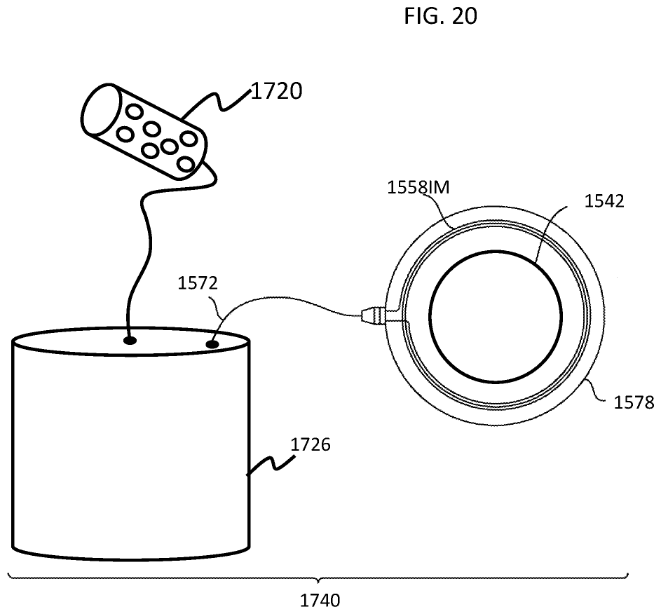

It is noted that while the embodiments detailed herein have focused on electrical detection/electrical monitoring/electrical analyses (ECE/EEG), other embodiments are related to detecting/monitoring, analyzing changes in the chemical composition of substances inside a body. By way of example only and not by way of limitation, FIG. 20 provides a schematic of an implantable component 1740 that is configured to monitor body fluid chemistry. In this regard, there is housing 1726 that includes a processor or the like that is program to analyze data via a signal from blood capture device 1720. The blood capture device 1720 is configured to capture blood and/or to analyze the blood to evaluate the chemistry thereof. By way of example only and not by way of limitation, the implantable component 1740 can be a blood glucose implant monitor that monitors blood directly or indirectly to determine its glucose level. The captured blood then is analyzed by a device 1726. Coil 1558IM can be a coil of a transcutaneous inductance apparatus 1578 as shown. This can be implantable the skin. The transcutaneous inductance apparatus includes a magnet 1540 to which can be utilized to hold an external transcutaneous inductance apparatus which may also include a magnet against the skin in general, and in alignment with the coil 1558IM in particular, so that the data obtained by the implants system, which is communicated via cable 1572 to the transcutaneous inductance apparatus, can be communicated to an external device such as a BTE device.

Note further that in an exemplary embodiment, the implantable component 1740 can be a new drug analyzer. By way of example only and not by way of limitation, the implantable component 1740 can be configured or otherwise programmed to analyze blood chemistry to evaluate the effects of a new drug.

The above said, it is noted that in at least some exemplary embodiments, an EEG system can be utilized to evaluate blood glucose levels and/or new drug efficacy. In this regard, there can be a scenario of use where there is a new drug introduction, and the evaluation regimen of the new drug introduction includes brain monitoring, where the brain monitoring includes application of an EEG monitoring. At least some of the exemplary embodiments detailed herein provide enablement for continuous monitoring, and such can be very utilitarian for new drug evaluation.

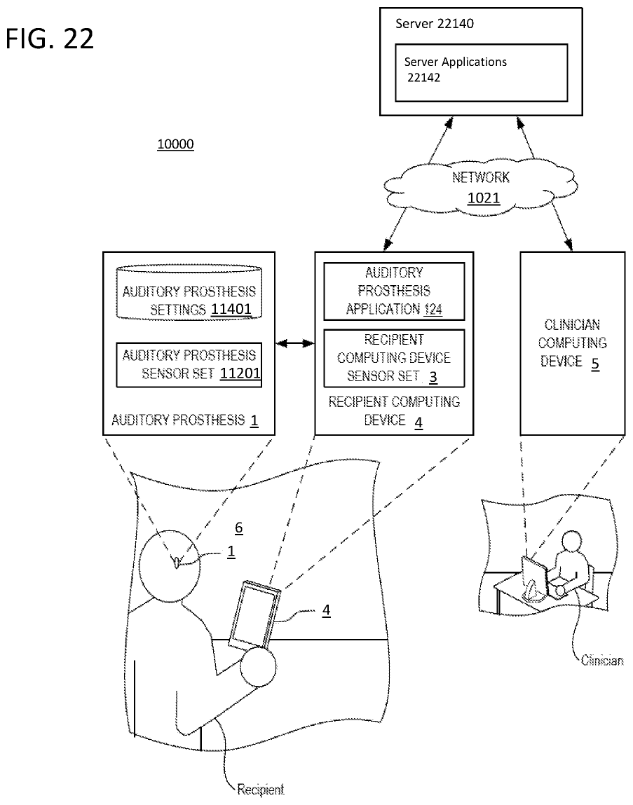

FIG. 22 illustrates an exemplar system 10000 that includes an auditory prosthesis 1 (this is representative of one embodiment, presented for textual economy—the auditory prosthesis is a proxy for any of the medical devices herein—any medical device can be substituted for the auditory prosthesis in some modified embodiments of the system of FIG. 22) that can be used with the technologies described herein. The system 10000 further includes a recipient (user of the medical device) computing device 120, a clinician computing device 5, and a server 22140, which are connected over a network 1021.

The network 1021 is a computer network, such as the Internet, that facilitates the communication of data among computing devices connected to the computer network.

As illustrated, the auditory prosthesis 1 (or whatever medical device disclosed herein is being used in the system of FIG. 22 in an alternate embodiment) and the recipient computing device 120 are operated by the recipient in an environment 6. The environment 6 defines the conditions in which the auditory prosthesis 1 and the recipient computing device 120 operate. In many examples herein, the environment 6 includes the sonic conditions in which the auditory prosthesis 1 functions. Such sonic conditions can include, for example, a loudness of noise (e.g., whether the environment 6 is loud or quiet), a number of sources of noise (e.g., a crowded restaurant with many sources of noise or a one-on-one conversation with fewer sources of noise) and a kind of noise (e.g., music or speech). The environment 6 can also define an activity in which the recipient is engaged, such as a conversation or exercise. The environment 6 can affect the operation of the auditory prosthesis 1, and the auditory prosthesis 1 can be customized to operate differently in different environments 6.

The auditory prosthesis 1 is a medical apparatus relating to a recipient's auditory system, such as a cochlear implant or bone conduction devices (e.g., percutaneous bone conduction devices, transcutaneous bone conduction devices, active bone conduction devices, and passive bone conduction devices), and middle ear stimulators, among others. The auditory prosthesis 1 can take any of a variety of forms. In the illustrated example, the auditory prosthesis includes an auditory prosthesis sensor set 11201 and operates according to auditory prosthesis settings 11401.

The auditory prosthesis sensor set 11201 is a collection of one or more hardware or software components of the auditory prosthesis 1 that obtain data, such as data regarding the environment 6, or the physiological data herein in the case of a body monitoring device, etc., the auditory prosthesis 1, or the recipient/user. In many examples, the auditory prosthesis sensor set 11201 include a microphone (e.g., an implanted or external microphone), which is a proxy for any monitoring device of a sensory medical device. The auditory prosthesis sensor set 11201 can include one or more other sensors, such as one or more accelerometers, gyroscopic sensors, location sensors, telecoils, biosensors (e.g., heart rate or blood pressure sensors), and light sensors, among others. The auditory prosthesis sensor set 11201 can include components disposed within a housing of the auditory prosthesis 1 (or whatever medical device at issue is applicable) as well as devices electrically coupled to the auditory prosthesis 1 (e.g., via wired or wireless connections). In examples, the auditory prosthesis sensor set 11201 includes a remote device connected to the auditory prosthesis 1 via an FM (Frequency Modulation) connection, such as a remote microphone (e.g., a COCHLEAR TRUE WIRELESS MINI MICROPHONE2+), a television audio streaming device, or a phone clip device, among other devices having FM transmission capabilities. The auditory prosthesis sensor set 11201 can further include sensors that obtain data regarding usage of the auditory prosthesis 1 (or other data identified herein), such as software sensors operating on the auditory prosthesis 1 that track: when the auditory prosthesis 1 is worn by the recipient, when the auditory prosthesis 1 (e.g., an external portion thereof) is removed from the recipient, when one or more of the auditory prosthesis settings 11401 are modified, and how long the auditory prosthesis 1 is operated using particular settings of the auditory prosthesis settings 11401, and/or the physiological data herein, among other data.

The auditory prosthesis settings 11401 are one or more parameters having values that affect how the auditory prosthesis 1 operates. For instance, the auditory prosthesis settings 11401 can include a map having minimum and maximum stimulation levels for frequency bands of stimulation channels. The map is then used by the auditory prosthesis 1 to control an amount of stimulation to be provided. For instance, where the auditory prosthesis 1 is a cochlear implant, the map affects which electrodes of the cochlear implant to stimulate and in what amount based on a received sound input. In some examples, the auditory prosthesis settings 11401 include two or more predefined groupings of settings selectable by the recipient. One of the two or more predefined groupings of settings may be a default setting.

The auditory prosthesis settings 11401 can also include sound processing settings that modify sound input before it is converted into a stimulation signal. Such settings can include, for example, particular audio equalizer settings can boost or cut the intensity of sound at various frequencies. In examples, the auditory prosthesis settings 11401 can include a minimum threshold for which received sound input causes stimulation, a maximum threshold for preventing stimulation above a level which would cause discomfort, gain parameters, loudness parameters, and compression parameters. The auditory prosthesis settings 11401 can include settings that affect a dynamic range of stimulation produced by the auditory prosthesis 1. As described above, many of the auditory prosthesis settings 11401 affect the physical operation of the auditory prosthesis 1, such as how the auditory prosthesis 1 provides stimulation to the recipient in response to sound input received from the environment 6.

The recipient computing device 120 is a computing device associated with the recipient of the auditory prosthesis 1. In many examples, the recipient computing device 120 is a cell phone, smart watch, or heart rate monitor, but can take other forms. Although described primarily in the context of the recipient, the recipient computing device 120 can be a computing device owned or primarily used by a parent or caregiver for the recipient. As illustrated, the recipient computing device 120 includes a recipient computing device sensor set 3.

The recipient computing device sensor set 3 is group of one or more components of the recipient computing device 120 that obtains data. The recipient computing device sensor set 3 can include one or more sensors, such as microphones, accelerometers, gyroscopic sensors, location sensors, biosensors (e.g., heart rate or blood pressure sensors), and light sensors, among others. The recipient computing device sensor set 3 can include components disposed within a housing of the recipient computing device 4 as well as devices electrically coupled to the recipient computing device 4 (e.g., via wired or wireless connections). In some examples, the recipient computing device sensor set 3 includes software sensors, such as software that obtains data from one or more data streams (e.g., audio streamed from the recipient computing device 4 to the auditory prosthesis 1). The recipient computing device sensor set 3 can further include sensors that obtain data regarding how the recipient computing device 4 itself is being used.

In examples, the recipient computing device 4 includes an auditory prosthesis application 124 that operates on the recipient computing device 4 and cooperates with the auditory prosthesis 1. The auditory prosthesis application 124 (or other medical device application 124) is a computer program stored as computer-executable instructions in memory on the recipient computing device 4 that, when executed, performs one or more tasks relating to the auditory prosthesis 1 or otherwise whatever medical device at issue is applicable. For instance, the auditory prosthesis application 124 can control the auditory prosthesis 1 (e.g., based on input received from the recipient), monitor usage of the auditory prosthesis 1, and obtain data from the auditory prosthesis 1. The recipient computing device 4 can connect to the auditory prosthesis 1 using, for example, a wireless radiofrequency communication protocol (e.g., BLUETOOTH). The auditory prosthesis application 124 transmits or receives data from the auditory prosthesis 1 over such a connection. The auditory prosthesis application 124 can also stream audio to the auditory prosthesis 1, such as from a microphone of the recipient computing device sensor set 3 or an application running on the recipient computing device 120 (e.g., a video or audio application). In examples, the auditory prosthesis application 124 functions as part of the recipient computing device sensor set 3 by obtaining data regarding the auditory prosthesis 1. The recipient computing device 120 can be in communication with one or both of the clinician computing device 5 and the server 22140, such as via the auditory prosthesis application 124 communicating over the network 1021.

The clinician computing device 5 is a computing device used by a clinician. A clinician is a medical professional, such as an audiologist, or a hearing prosthesis device technician but any disclosure herein of an audiologist, or other medical professional corresponds to an alternative disclosure of another embodiment where the person is engaged in the outfitting or otherwise provisioning of a hearing prosthesis. In an example, the clinician is a medical professional that provides care or supervision for the recipient. In an example, the clinician is a proxy for another embodiment of a person that provides or otherwise works with hearing prostheses. The clinician computing device 5 includes one or more software programs usable to monitor or control the auditory prosthesis 1, such as customization of the auditory prosthesis settings 11401.

The server 22140 is a server remote from the auditory prosthesis 1, recipient computing device 120, and the clinician computing device 5. The server 22140 is communicatively coupled to the recipient computing device 120 and the clinician computing device 5 via the network 1021. In many examples, the server 22140 is indirectly communicatively coupled to the auditory prosthesis 1 through the recipient computing device 120 (e.g., via the auditory prosthesis application 124). In some examples, the server 22140 is directly communicatively coupled to the auditory prosthesis 1. The server 22140 includes one or more server applications 22142.

The one or more server applications 22142 are computer programs stored as computer-executable instructions in memory on the server 22140 that, when executed, perform one or more tasks relating to the system 10000. The one or more server applications 22142 are operable to perform one or more operations described herein, such as operations that customize the auditory prosthesis 1. As illustrated, the one or more server applications 22142 operate on the server 22140.

The components of the system 10000 can cooperate to perform a method that improves the performance of the auditory prosthesis 1.

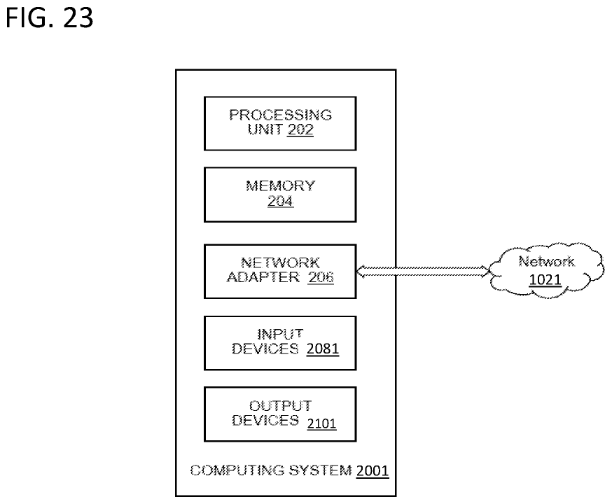

FIG. 23 illustrates an example of a suitable computing system 2001 with which one or more of the disclosed examples can be implemented, where one or more or all of the disclosed method actions herein that are automated or otherwise computer-based can be implemented utilizing the arrangement of FIG. 23. Computing systems, environments, or configurations that can be suitable for use with examples described herein include, but are not limited to, personal computers, server computers, hand-held devices, laptop devices, multiprocessor systems, microprocessor-based systems, programmable consumer electronics (e.g., smart phones), network PCs, minicomputers, mainframe computers, tablets, distributed computing environments that include any of the above systems or devices, and the like. The computing system 2001 can be a single virtual or physical device operating in a networked environment over communication links to one or more remote devices. The remote device can be an auditory prosthesis (e.g., the auditory prosthesis 1), a personal computer, a server, a router, a network personal computer, a peer device or other common network node. In examples, the recipient computing device 120, the clinician computing device 5, and the server 22140 includes one or more components or variations of components of the computing system 2001. Further, in some examples, the auditory prosthesis 1 includes one or more components of the computing system 2001.

In its most basic configuration, computing system 2001 includes at least one processing unit 20222 and memory 20444.

The processing unit 20222 includes one or more hardware or software processors (e.g., Central Processing Units) that can obtain and execute instructions. The processing unit 20222 can communicate with and control the performance of other components of the computing system 2001. The memory 20444 is one or more software- or hardware-based computer-readable storage media operable to store information accessible by the processing unit 20222. The memory 20444 can store, among other things, instructions executable by the processing unit 20222 to implement applications or cause performance of operations described herein, as well as other data. The memory 20444 can be volatile memory (e.g., RAM), non-volatile memory (e.g., ROM), or combinations thereof. The memory 20444 can include transitory memory or non-transitory memory. The memory 20444 can also include one or more removable or non-removable storage devices. In examples, the memory 20444 can include RAM, ROM, EEPROM (Electronically-Erasable Programmable Read-Only Memory), flash memory, optical disc storage, magnetic storage, solid state storage, or any other memory media usable to store information for later access. In examples, the memory 20444 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 20444 can include wired media such as a wired network or direct-wired connection, and wireless media such as acoustic, RF, infrared and other wireless media or combinations thereof.

In the illustrated example, the system 2001 further includes a network adapter 20666, one or more input devices 2081, and one or more output devices 2101. The system 2001 can include other components, such as a system bus, component interfaces, a graphics system, a power source (e.g., a battery), among other components.

The network adapter 20666 is a component of the computing system 2001 that provides network access. The network adapter 20666 can provide wired or wireless network access and can support one or more of a variety of communication technologies and protocols, such as ETHERNET, cellular, BLUETOOTH, near-field communication, and RF (Radiofrequency), among others. The network adapter 20666 can include one or more antennas and associated components configured for wireless communication according to one or more wireless communication technologies and protocols.

The one or more input devices 2081 are devices over which the computing system 2001 receives input from a user. The one or more input devices 2081 can include physically-actuatable user-interface elements (e.g., buttons, switches, or dials), touch screens, keyboards, mice, pens, and voice input devices, among others input devices.

The one or more output devices 2101 are devices by which the computing system 2001 is able to provide output to a user. The output devices 2101 can include, displays, speakers, and printers, among other output devices.

It is noted that any method action disclosed herein corresponds to a disclosure of a non-transitory computer readable medium that has program there on a code for executing such method action providing that the art enables such. Still further, any method action disclosed herein where the art enables such corresponds to a disclosure of a code from a machine learning algorithm and/or a code of a machine learning algorithm for execution of such. Still as noted above, in an exemplary embodiment, the code need not necessarily be from a machine learning algorithm, and in some embodiments, the code is not from a machine learning algorithm or the like. That is, in some embodiments, the code results from traditional programming. Still, in this regard, the code can correspond to a trained neural network. That is, as will be detailed below, a neural network can be “fed” significant amounts (e.g., statistically significant amounts) of data corresponding to the input of a system and the output of the system (linked to the input), and trained, such that the system can be used with only input, to develop output (after the system is trained). This neural network used to accomplish this later task is a “trained neural network.” That said, in an alternate embodiment, the trained neural network can be utilized to provide (or extract therefrom) an algorithm that can be utilized separately from the trainable neural network. In one embodiment, there is a path of training that constitutes a machine learning algorithm starting off untrained, and then the machine learning algorithm is trained and “graduates,” or matures into a usable code—code of trained machine learning algorithm. With respect to another path, the code from a trained machine learning algorithm is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice some of the method actions, and thus are not present the ultimate system. Instead, only the resulting product of the learning is used.

An exemplary system includes an exemplary device/devices that can enable the teachings detailed herein, which in at least some embodiments can utilize automation. That is, an exemplary embodiment includes executing one or more or all of the methods detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A system, comprising:
a body worn and/or implanted medical device, wherein
the medical device is configured to receive and store data indicative of past treatment of a person who is the wearer and/or has been implanted with the medical device, and
the medical device is configured to at least one of provide tissue stimulation to tissue of the person or sense phenomenon associated with the person.

2. The system of claim 1, wherein:
the medical device is a first subsystem of the system;
the system includes a second subsystem;
the medical device is configured to electronically communicate data indicative of the stored data to the second subsystem; and
the second subsystem is configured to receive the communicated data and at least one of provide the data in a human understandable format or analyze the data and develop at least a partial treatment regimen based on the analysis.

3. The system of claim 2, wherein:
the second subsystem is configured to analyze the data and develop at least a partial treatment based on the recipient;
the second subsystem is configured to communicate the developed at least partial treatment to the medical device; and
the medical device is configured to receive the communicated developed at least partial treatment and operate in a different manner based on the received communicated developed at least partial treatment.

4. (canceled)

5. The system of claim 1, wherein:
the medical device is an implantable hearing prosthesis.

6-8. (canceled)

9. The system of claim 1, wherein:
the medical device has stored therein data indicative of therapeutic substance(s) taken by the person within at least the past 10 days.

10. The system of claim 1, wherein:
the medical device is configured to identify biomarkers of the person and determine a compliance of past treatment.

11. A method, comprising:
receiving, indirectly or directly, from a body worn and/or implanted medical device, data indicative of at least one of a treatment history or a current medical condition of a person wearing and/or implanted with the medical device;
evaluating at least the data and determining, based on the evaluation, a treatment regimen for the person; and
implementing the treatment regimen or providing the treatment regimen to the person.

12. The method of claim 11, wherein:
the body worn and/or implanted medical device is configured to sense a physiological phenomenon of the person, and at least prior to the action of receiving, has stored therein the data indicative of the treatment history of the person;
the person is completely conscious; and
the action of evaluating is executed without real time input from the person regarding the treatment history.

13. The method of claim 11, wherein:
the body worn and/or implanted medical device is configured to sense a physiological phenomenon of the person, and at least prior to the action of receiving, has stored therein the data indicative of the treatment history of the person; and
the action of evaluating and determining is executed automatically using artificial logic.

14-19. (canceled)

20. The method of claim 11, wherein:
the medical device is a hearing prosthesis;
the hearing prosthesis was used in conjunction with the person to evaluate the person's hearing; and
the data is indicative of a current medical condition, wherein the current medical condition is the person's ability to hear, and the data is based on data obtained when using the hearing prosthesis to evaluate the person's hearing.

21. The method of claim 20, wherein:
the treatment regimen is the adjustment and/or the addition of a therapeutic substance to treat hearing disorder related ailments.

22. The method of claim 11, wherein:
the person has been taking a therapeutic substance to treat an ailment for at least a month prior to the action of determining; and
the action of evaluating and determining results in a treatment regimen for the person that results in a decrease in an average daily amount of the therapeutic substance during the time of implementing the treatment regimen.

23. The method of claim 11, wherein:
the body worn and/or implanted medical device is configured to sense a physiological phenomenon of the person;
the method further includes implementing the treatment regimen automatically;
the person has a therapeutic substance delivery system attached thereto;
the action of implementing the treatment regimen automatically causes the therapeutic substance delivery system to automatically provide a fast acting therapeutic substance in a quantity and/or quality automatically determined in the action of determining the treatment regime; and
for at least two weeks prior to the action of implementing the treatment regimen, the person has been receiving therapeutic substance in amounts and/or types substantially different from that provided during the action of automatically providing.

24. A closed-loop monitoring and therapeutic substance delivery system, wherein:
the closed-loop monitoring and therapeutic substance delivery system is configured to monitor one or more physiological parameters of a user of a medical device; and
the closed-loop monitoring and therapeutic substance delivery system is further configured to use results from the monitored one or more physiological parameters to establish a dosage and/or schedule for future therapeutic substance(s) to be taken by and/or administered to the user.

25. The closed-loop monitoring and therapeutic substance delivery system of claim 24, wherein:
the closed-loop monitoring and therapeutic substance delivery system is further configured to have access to data indicative of therapeutic substance(s) previously used and/or currently used by the user; and
the closed-loop monitoring and therapeutic substance delivery system is further configured to use results from the monitored one or more physiological parameters in conjunction with the data indicative of therapeutic substance(s) to establish the dosage and/or schedule for future therapeutic substance(s) to be taken by and/or administered to the user.

26. The system of claim 24, wherein:
the system includes an implanted and/or body worn component that monitors the one or more physiological parameters; and
the system further includes a therapeutic substance delivery device to deliver the future therapeutic substance(s).

27. The system of claim 26, wherein:
the system includes a first subsystem, the first subsystem being configured to execute the monitoring and obtaining of the data; and
the delivery device is in signal communication with the first subsystem and/or another subsystem in signal communication with the first subsystem and wherein the delivery device is configured to receive data based at least in part on data based on the dosage and/or schedule and operate accordingly to deliver the future therapeutic substance(s) to the recipient.

28. The system of claim 27, wherein:
the delivery device is configured to operate accordingly to deliver the therapeutic substance(s) according to the dosage and/or schedule only if the recipient approves the operation.

29. The system of claim 26, wherein:
the system includes a first subsystem, the first subsystem being configured to execute the monitoring and obtaining of the data; and
at least the first subsystem or a second subsystem that is a part of the system and is in signal communication with the first subsystem is configured to provide output indicative of the dosage and/or schedule; and
the delivery device is configured to enable manual initiation of dispensement of the therapeutic substance based on the output.

30. The system of claim 24, wherein:
the system includes a monitoring device configured to monitor the one or more physiological parameters; and
the system includes a therapeutic substance delivery device configured to deliver therapeutic substance to the user, the therapeutic substance delivery device being a physically separate device from the monitoring device.

31-41. (canceled)
